(19) United States
(12) Patent Application Publication
(10) Pub. No.: US 2015/0351851 A1
(43) Pub. Date: Dec. 10, 2015

(54) Blood Pressure Cuff Shield Incorporating Antimicrobial Technology

(71) Applicant: EASTERN MAINE HEALTHCARE SERVICES, Brewer, ME (US)

(72) Inventors: Claire T. Deselle, Sebago, ME (US); Kerem Durdag, Scarborough, ME (US); Brittany Paul, Old Orchard Beach, ME (US); Valerie Gunn, Cape Neddick, ME (US); Brian Pendleton, Newcastle, ME (US); Richard Provonchee, Cushing, ME (US)

(21) Appl. No.: 14/762,775

(22) PCT Filed: Feb. 24, 2014
(86) PCT No.: PCT/US14/18002

§ 371 Date: Jul. 22, 2015

Related U.S. Application Data
(60) Provisional application No. 61/767,928, filed on Feb. 22, 2013.

(51) Int. Cl. A61B 19/08; A61B 5/022

(52) U.S. Cl. CPC A61B 19/088; A61B 19/081; A61B 5/022; A61B 2019/086; A61B 2562/247

(57) ABSTRACT

Disclosed is a an economical blood pressure cuff shield that acts as an hygienic barrier between patients and blood pressure cuffs during blood pressure measurement. The shield includes antimicrobial properties, which eradicate microorganisms on contact, protecting the shield from communicated pathogens and preventing the blood pressure cuff from colonization of the common health-care associated microbes. With the antimicrobial properties, the shield can be used for multiple patients over a 24 hour period. After the indicated period of use, the blood pressure cuff shield is removed and discarded. The biodegradable and cost effective construction allows the health care field to stride towards eco-friendly solutions to improve sanitation and sterility of facilities.

### FIELD OF INVENTION

This field of invention relates to a disposable healthcare product and material whereby a low bioburden biodegradable and/or compostable absorbent nonwoven medium which does not support bacterial growth is employed in conjunction with at least one antimicrobial agent such as silver-based and/or silver ion-based active ingredients in the absorbent media or other consumable item which attaches to a blood pressure cuff. The disposable, consumable, healthcare blood pressure cuff covering/shield product material of the present invention functions to destroy microbes as they come into contact with the blood pressure cuff covering material itself thereby risk mitigating the spread of pathogens to the blood cuff itself and/or from patient to patient. Active ingredients that are part of the blood pressure cuff covering/shield material of the present invention can function in the condensed phase and the biodegradable nonwoven material can function as a carrier and/or a release vehicle for one or more antimicrobial and/or antifungal chemicals or other actives. Further, the product material described herein comprises conformability, comfort, and ease of application and removal.

### BACKGROUND OF THE INVENTION

Sanitation and sterility of health-care environments is critical in reducing the transmission of health-care associated infections. The CDC estimates that 5% of patients admitted into a hospital are likely to acquire an infection while receiving care, culminating in 1.7 million infections and approximately 99,000 deaths each year (Healthcare Associated Infections in North Carolina, N.C. Department of Health and Human Services, 2012). Sanitization in Intensive Care Units is of extreme importance as 51% of patients in ICUs worldwide that have infections are more than twice as likely to die compared to patients without infections (JAMA, 2009; 302(21)). It has been found that 80% of infectious diseases are transferred by touch (based on our knowledge-base and conversations with health care professionals).

A worldwide study published in the Journal of the American Medical Association surveyed the infection status of over 13,000 patients from 1,200 Intensive Care Units (ICUs) in 75 countries. The survey found that more than half of all patients had an infection and those that were infected were more than twice as likely to die as uninfected patients. In addition to increased mortality, it was found that the risk for acquiring an infection increases the longer a patient stays in the ICU. Of those patients that were in the ICU for a day or less, only 32% had infections, while 70% of those patients that stayed in the ICU for more than a week had infections (JAMA, 2009; 302(21)).

While healthcare professionals employ strict infection control measures including hand-washing and frequent surface disinfection, these measures are not enough as the number of hospital acquired infections each year continues to rise (JAMA, 2009; 302(21)). Frequently touched surfaces in ICUs are heavily contaminated with anywhere from several hundred to over ten thousand colony forming units of infectious bacteria. These surfaces are touched by patients, families, doctors, nurses, and cleaning staff and it is exactly here where an added line of defense, specifically in the blood cuff application, is needed.

Upon admission into a medical facility (i.e. hospital, physician's office) it is common practice to obtain the blood pressure of each individual patient using a sphygmomanometer. The blood pressure cuff is secured around a patient's arm, usually in contact with bare skin which is colonized with a plethora of microorganisms including, but not limited to, Methicillin Resistant Staphylococcus aureus (MRSa), E. coli, Vancomycin-resistant Enterococcus (VRE) and P. aeruginosa.

The capacity of blood pressure cuffs to act as vehicles of hospital infection has been recognized (The microbial flora of in-use blood pressure cuffs, .Ir J Med Sci. 1991 April; 160(4):112-3). In an important study, blood pressure cuffs from various inpatient settings were found to have bacterial colonization rates of 81-100%. Also, 45.7% of the “clean” cuffs were contaminated with organic and/or inorganic substances. The patient contact sides of cuffs were contaminated twice as often as the non-patient sides (Stemicht A L. Significant bacterial colonization of the surface of non-disposable sphygmomanometer cuffs and reused disposable cuffs. Comet Med. Ctr., New York, N.Y. 10021).This followed one of several studies such as the peer-reviewed report of 18 hospital units that “revealed” a level of contamination reaching 100 or more colony-forming units per 25 cm² was observed on 92 (45%) of inner sides and 46 (23%) of outer sides of 203 cuffs. The highest rates of contamination occurred on the inner side of BP cuffs kept in intensive care units (ICUs) (20 [83%] of 24) or on nurses' trolleys (27 [77%] of 35). None of the 18 BP cuffs presumed to be clean (i.e., those that had not been used since the last decontamination procedure) had a high level of contamination. Potentially pathogenic microorganisms were isolated from 27 (13%) of the 203 BP cuffs: 20 of these microorganisms were Staphylococcus aureus, including 9 methicillin-resistant strains. The highest rates of contamination with potentially pathogenic microorganisms were observed on cuffs used in ICUs and those kept on nurses' trolleys.” (Blood Pressure Cuff as a Potential Vector of Pathogenic Microorganisms: A Prospective Study in a Teaching Hospital, Infection Control and Hospital Epidemiology, September 2006, vol. 27, no. 9).

In attempts to reduce transmission of health-care associated infections from patient to patient via blood pressure cuffs, a few solutions have been tried within the medical industry, but have proven unsuccessful. Single patient, disposable blood pressure cuffs are at this time being utilized within numerous health-care facilities, but not as indicated. Blood pressure cuffs designed for single patient use, due to high cost, are currently being used for extended periods of time over multiple-patients.

Sanitizing wipes are the chosen disinfection method, used widely throughout the health-care field. The various wipes have shown efficacy against a broad range of microorganisms, under the proposed indications for use, which typically include a lengthy contact time (up to 10 minutes). For most disinfectants, including combinations of bleach, isopropyl alcohol, quaternary amines, ethanol, and phenol, the contact time out lasts the time it takes for the product to dry, which would require continuous application of the disinfectant over an extended period of time. The time necessary for proper sanitation is rarely given and staffing limitations often lead to inappropriate use of disinfectants (Technical Bulletin on Contact Times, Virox).

Current art for blood pressure cuff protective devices includes that of blood pressure cuff liners, barriers, and guards. Some liners/guards simply attach to the blood pressure cuff, while others envelop the entirety of the blood pressure cuff. All of which do not indicate multi-patient use and must be applied and removed between patient to remain an effective method. Blood pressure cuff barriers, intended to be applied to a patient's arm, come in the form of sleeves, wraps, etc. All above devices are intended for single patient application and incur significant cost and present time burden upon medical staff.

Described herein, a state of the art blood pressure cuff shield, which is applied to the blood pressure cuff, acts as a barrier between the patient and the cuff, preventing the spread of communicable pathogens to the blood pressure cuff. Integrated antimicrobial agents mitigate microorganisms on contact with the shield, allowing for multi-patient use of a single shield over a 24 hour period. The blood pressure cuff shield will be a cost effective and time efficient method that hospitals will integrate into their disinfection practices in order to promote cleanliness and sterility of the facility. Exemplary representation of the current art of blood pressure cuff protection is provided below.

### Description of Related Art

### Blood Pressure Cuff Liners/Shields

Various blood pressure cuff protective devices have been patented in a range of structures including: arm sleeves and arm wraps to be applied to the appendage of the patient, and envelopes and liners that are applied to the blood pressure cuff itself. All of which function in an equivalent manner, preventing transmission of pathogens while protecting the blood pressure cuff from bacterial colonization. While most devices are made for single patient use, antimicrobial agents can be utilized to eradicate pathogens on contact allowing for a reusable product. Biodegradability of the disposable protective devices is a significant advantage as the world transitions to eco-friendly consumables.

U.S. Pat. No. 5,513,643 teaches a disposable protective wrap that comprises a flexible, nonporous material and fastening devices. The device is rectangular and sufficient in length to allow coverage of limb circumference. The wrap is to be secured around the limb and the sphygmomanometer is applied over the wrap. Acting as a barrier between the patient and the blood pressure cuff, the protective wrap prevents exposure of microbes and other contaminants which may be transferred to the blood pressure cuff. According to the disclosure, the nonporous material can be composed of polyethylene, rayon acetate, vinyl or other flexible non-porous materials. The fasteners may include hook and loop, self-adhering materials, or buttons. Though the invention provides protection to the blood pressure cuff from transfer of pathogens, it does not demonstrate multiple patient uses. A single wrap must be applied to each and every patient being admitted to a medical facility, which would prove to be timely and costly. Further, the protective device is not biodegradable and does not provide an antimicrobial agent. The current invention incorporates antimicrobial agents into a non-woven melt-blown fiber created from polylactic acid (PLA), that is 100% biodegradable and which is not taught within the prior art.

U.S. Pat. No. 5,620,001 describes a universal blood pressure cuff cover system consisting of a top and bottom band. The top band is received about the limb of the patient where the top band is received about the limb and the blood pressure cuff. The bottom band is comprised of a two ply material, preferably spun-bond polypropylene. The first layer is soft and absorbent which engages with the skin, where the second layer is a thin, soft, and flexible fluid impervious plastic film. The blood pressure cuff is secured over the bottom band and the top band is applied over the cuff. A light, flexible, fluid impermeable material comprises the top band. The described blood pressure cuff cover, again, is designed for single patient use and will prove cumbersome to health-care providers. The prior art demonstrates a device that had two parts, one that fits to the patient's appendage and one that covers the blood pressure cuff once secured to the patient's appendage. The current invention provides a simpler solution wherein the hygienic barrier is applied to the blood pressure cuff for multi-patient use. There is no mention of biodegradability and/or incorporation of antimicrobial properties of the prior art. The substrate used in the prior art differs from that of the current invention by choice of polymer and process used to manufacture the substrate. The prior art utilizes a spun-bond process to manufacture poly-propylene fabric wherein the current art PLA is manufactured by way of a melt-blown extrusion process.

U.S. Pat. No. 6,525,238 discloses a single use disposable skin and cuff protector in the form of a wrap that is applied to the limb of the patient. The hygienic barrier provides protection to the patient and the blood pressure cuff from pathogens being communicated from one to another. The wrap is composed of a non-porous material such as polyethylene layered with a soft woven or non-woven absorbent layer. It is rectangular in shape and the width extends past the blood pressure cuff by two inches. The wrap is secured by a non-reusable fastener such as adhesive to ensure that the wrap is only used a single time. No indication was made of incorporated antimicrobials with the described art. According to the disclosure, the invention is specifically designed for single patient application. Finally, the patent does not disclose biodegradability of the substrates used for the hygienic barrier. The substrate used within the current invention differs from the prior art in that a PLA non-woven manufactured by a melt-blown extrusion process is used and the antimicrobial agents are imbedded within the melt-blown fibers.

U.S. Patent Publication No. 2004/0049114 A1, assigned to Ethox Corporation, demonstrates a reusable/disposable enclosure a for blood pressure cuff. The enclosure or envelope comprises a pouch in which the blood pressure cuff is inserted. The pouch is sealed around the blood pressure cuff by flexible zipper seal. The enclosure is described as being constructed from at least one ply of translucent polyolefin composed of a spun non-woven skin contact surface and a non-porous, liquid impermeable inner surface. Hook and loop fasteners are positioned at terminal ends of the enclosure in lieu of the covered fasteners on the blood pressure cuff. The indicated purpose for this invention is to prevent a contaminated blood pressure cuff from contacting a patient and to cover unattractive and/or soiled blood pressure cuffs. Within the discussion of the art, no consideration is made for the incorporation of antimicrobial properties. Further, the description of the art does not discuss biodegradability or biocompostibility of the product. Lastly, the current art differs from the prior art in that a PLA non-woven substrate with imbedded antimicrobials is manufactured by way of a melt-blown extrusion process.

### Description of Related Art

### Antimicrobial/Antifungal Blood Pressure Cuff Liner/Shield

U.S. Patent Publication No. 2010/0089408 A1, assigned to Goodwin Procter LLP, discloses a multifaceted blood pressure cuff liner that can be applied around a patient's arm or directly to the blood pressure cuff. The liner may be composed of 3 ply tissue paper, non-woven spun bond polypropylene, or a laminate comprised of 2 ply tissue with polyethylene. The liner is rectangular in shape and is as wide as or slightly wider than the blood pressure cuff. In the application of using the liner at a wrap, adhesive strips may be applied to the short end of the liner. The author described that various fastening systems can be utilized in order to secure the liner to the patient's arm; including but not limited to a hook and loop system, buttons, and snaps. As a liner applied to the blood pressure cuff, an adhesive strip running length wise down the center of the apparatus secures the liner to the blood pressure cuff. U.S. Patent Publication No. 2010/0089408 A1 teaches that the liner may, also, be secured to the blood pressure cuff by medical tape, clips, or a hook and loop system. The patent discloses use of antimicrobial agents such as organosilane, ionic silver, or silver nano-particles. The methods used to coat the liner substrate with silver include, vacuum sputter coating and plasma arc deposition to apply vaporized silver, and ionic plasma deposition of silver oxides for production of silver ions. Although this prior art claims antimicrobial coatings, it does not include any teachings for the incorporation of silver zeolite technology within the fibers of the substrate of the liner as an antimicrobial agent. The prior art includes the use of a spun-bond polypropylene non-woven, where the non-woven used within the current invention is manufactured from PLA by way of a melt-blown extrusion process.

### Description of Related Art

### Fibers Containing Antimicrobial Agents Inclusive of Copper and Silver

U.S. Patent Publication No. 20120164449 describes a synthetic fiber comprising: a polymer; an antimicrobial agent; and a dispersion liquid, wherein the dispersion liquid impregnates the fibers. The claimed antimicrobial agents include silver, copper, zinc, gold, or a combination thereof, in the metallic form, salt form, or ionic form. The dispersion liquid used is selected from a group consisting of anti-stat, ionic anti-stat oil, phosphate ester, wax, and vegetable oil. The composition of prior art differs from the present invention of which the antimicrobial agents consisting of silver and copper zeolite are imbedded within the fibers of the non-woven material. No dispersion liquid is necessary for applying the antimicrobials to the present invention, instead the antimicrobials are master batched within the polymer and dispersed through melt-blown processing. Though the prior art claims the polymer can be processed through spinnerets, it does not specifically teach the art of melt-blown processing of polymers; specifically PLA with imbedded antimicrobial agents.

U.S. Patent Publication No. 20100227052, assigned to Baxter, teaches a method of processing for a substrate having a coating comprising a metal and exposing the substrate surface to a halogen-containing gas. The substrate surface comprises a plastic or elastomer selected from the group consisting of: acrylonitrile butadiene styrenes, polyacrylonitriles, polyamides, polycarbonates, polyesters, polyetheretherketones, polyetherimides, polyethylenes, polyethylene terephthalates, polylactic acids, polymethyl methacrylates, polypropylenes, polystyrenes, polyurethanes, poly(vinyl chlorides), polyvinylidene chlorides, polyethers, polysulfones, silicones, natural rubbers, synthetic rubbers, styrene butadiene rubbers, ethylene propylene diene monomer rubbers, polychloroprene rubbers, acrylonitrile butadiene rubbers, chlorosulphonated polyethylene rubbers, polyisoprene rubbers, isobutylene-isoprene copolymeric rubbers, chlorinated isobutylene-isoprene copolymeric rubbers, brominated isobutylene-isoprene copolymeric rubbers, and blends and copolymers thereof. The metals used for coatings include: silver, copper, gold, zinc, cerium, platinum, palladium, tin, or mixtures thereof. The method described therein differs from the present invention by way of the antimicrobial integration. The prior art describes coating the substrate surface with an antimicrobial agent, whereas the current invention has the antimicrobial integrated within/imbedded into the fibers. The prior art does not mention the use of melt-blown non-woven fabric, nor does it teach the manufacturing of a melt-blown non-woven fiber as in the current invention.

U.S. Patent Publication No. 20090324738, assigned to Baxter, discloses a method for forming an antimicrobial coating on a substrate surface composed of a mixture comprising a transition metal, a biguanide compound, and a reducing agent, wherein the mixture is free of polymeric binders. The mixture is deposited onto the surface of the substrate, thereby forming a coated substrate surface. The substrate surface comprises a plastic or elastomer selected from the group consisting of acrylonitrile butadiene styrenes, polyacrylonitriles, polyamides, polycarbonates, polyesters, polyetheretherketones, polyetherimides, polyethylenes, polyethylene terephthalates, polylactic acids, polymethyl methacrylates, polypropylenes, polystyrenes, polyurethanes, poly(vinyl chlorides), polyvinylidene chlorides, polyethers, polysulfones, silicones, natural rubbers, synthetic rubbers, styrene butadiene rubbers, ethylene propylene diene monomer rubbers, polychloroprene rubbers, acrylonitrile butadiene rubbers, chlorosulphonated polyethylene rubbers, polyisoprene rubbers, isobutylene-isoprene copolymeric rubbers, chlorinated isobutylene-isoprene copolymeric rubbers, brominated isobutylene-isoprene copolymeric rubbers, and blends and copolymers thereof. The chosen antimicrobial agents include silver, copper, gold, zinc, cerium, platinum, palladium, tin, and mixtures thereof. Again, the prior art describes coating the substrate surface with an antimicrobial agent, whereas the current invention has the antimicrobial integrated within/imbedded into the fibers. The method for integration of the antimicrobials within the current invention includes pelletizing the copper and silver antimicrobial agent into the polymer resin that will be processed by way of melt-blown extrusion. Lastly, the prior art differs from the current invention in that the antimicrobial agent used is a metal salt, whereas the imbedded antimicrobial agent of the current invention is a silver and copper zeolite.

U.S. Patent Publication No. 20090324666, assigned to Baxter, defines a method for forming an antimicrobial resin composed of a mixture comprising about 15% weight to 80% weight of a hydrophilic acrylic oligomer, about 10% weight of a multifunctional acrylic monomer, about 5% weight to about 40% weight of an adhesion promoting acrylic or vinyl monomer, and about 0.1% weight to about 15% weight of an antimicrobial metal salt, and exposing the mixture to a radiation source to cure at least a portion of the mixture, thereby forming an antimicrobial resin. The method claims providing the mixture on a substrate before exposing the mixture to the radiation source. Substrates surfaces described within the prior art include: a plastic or elastomer selected from the group consisting of acrylonitrile butadiene styrenes, polyacrylonitriles, polyamides, polycarbonates, polyesters, polyetheretherketones, polyetherimides, polyethylenes, polyethylene terephthalates, polylactic acids, polymethyl methacrylates, polypropylenes, polystyrenes, polyurethanes, poly(vinyl chlorides), polyvinylidene chlorides, polyethers, polysulfones, silicones, natural rubbers, synthetic rubbers, styrene butadiene rubbers, ethylene propylene diene monomer rubbers, polychloroprene rubbers, acrylonitrile butadiene rubbers, chlorosulphonated polyethylene rubbers, polyisoprene rubbers, isobutylene-isoprene copolymeric rubbers, chlorinated isobutylene-isoprene copolymeric rubbers, brominated isobutylene-isoprene copolymeric rubbers, and blends and copolymers thereof. The chosen antimicrobial agents include silver, copper, gold, zinc, cerium, platinum, palladium, tin, and mixtures thereof. The prior art is different from the current invention by the method of which is used to impregnate the substrate with antimicrobial agents. The prior art describes a coating and curing process, where the current invention describes a masterbatch and melt-blown extrusion method for imbedding the antimicrobials into fibers of the substrate. Further, the prior art differs from the current invention in that the antimicrobial agent used is a metal salt, whereas the imbedded antimicrobial agent of the current invention is a silver and copper zeolite. Lastly, the prior art does not teach the use of melt-blown non-woven fabric as the substrate surface, where the current invention teaches the manufacturing of said melt-blown non-woven incorporating antimicrobial agents.

U.S. Patent Publication No. 200903317435, assigned to Baxter, describes a method for processing a substrate having a coating comprising a metal, exposing it to an oxidizing agent and an anion. The claimed substrate may be comprised of the following: a plastic or elastomer selected from the group consisting of acrylonitrile butadiene styrenes, polyacrylonitriles, polyamides, polycarbonates, polyesters, polyetheretherketones, polyetherimides, polyethylenes, polyethylene terephthalates, polylactic acids, polymethyl methacrylates, polypropylenes, polystyrenes, polyurethanes, poly(vinyl chlorides), polyvinylidene chlorides, polyethers, polysulfones, silicones, natural rubbers, synthetic rubbers, styrene butadiene rubbers, ethylene propylene diene monomer rubbers, polychloroprene rubbers, acrylonitrile butadiene rubbers, chlorosulphonated polyethylene rubbers, polyisoprene rubbers, isobutylene-isoprene copolymeric rubbers, chlorinated isobutylene-isoprene copolymeric rubbers, brominated isobutylene-isoprene copolymeric rubbers, and blends and copolymers thereof. The metals include: silver, copper, gold, zinc, cerium, platinum, palladium, tin, and mixtures thereof. The product of the described prior art differs from the current invention by way of antimicrobial integration of the substrate. Again, the antimicrobial agents of the current invention are integrated within the fibers of the substrate itself rather than coated. The integration process for the current invention does not include a secondary process exposing the substrate to and oxidizing agent or anions. Also, the prior art does not teach the use of melt-blown non-woven fabric as the substrate, nor does it mention the method of manufacturing said melt-blown non-woven fabric.

U.S. Patent Publication No. 200903317435, assigned to Baxter, teaches a method for processing a substrate comprising metallic nanoparticles and exposing the substrate surface to a plasma. The substrate surface may include: a plastic or elastomer selected from the group consisting of acrylonitrile butadiene styrenes, polyacrylonitriles, polyamides, polycarbonates, polyesters, polyetheretherketones, polyetherimides, polyethylenes, polyethylene terephthalates, polylactic acids, polymethyl methacrylates, polypropylenes, polystyrenes, polyurethanes, poly(vinyl chlorides), polyvinylidene chlorides, polyethers, polysulfones, silicones, natural rubbers, synthetic rubbers, styrene butadiene rubbers, ethylene propylene diene monomer rubbers, polychloroprene rubbers, acrylonitrile butadiene rubbers, chlorosulphonated polyethylene rubbers, polyisoprene rubbers, isobutylene-isoprene copolymeric rubbers, chlorinated isobutylene-isoprene copolymeric rubbers, brominated isobutylene-isoprene copolymeric rubbers, and blends and copolymers thereof. The chosen metallic nanoparticles include: silver, copper, gold, zinc, cerium, platinum, palladium, tin, and mixtures thereof. The processing of the prior art significantly differs from the current invention. As stated above, the current invention processed coated metallic substrate surfaces with plasma, whereas the current invention incorporates the silver and copper zeolite through melt-blown manufacturing of substrate. The current invention differs from the prior art in which the PLA is melt-blown into a non-woven fabric.

U.S. Patent Publication No. 20060121078 teaches a medical device having antimicrobial properties comprising a matrix polymer having dispersed particles of a hydrophilic powder having an encapsulated or dispersed antimicrobial metal or metal ion containing inorganic antimicrobial agent capable of releasing said antimicrobial metal or metal ion. Said hydrophilic polymer having water absorption at equilibrium of at 5% by weight, wherein the rate of release of the antimicrobial metal or metal ions is limited by or regulated by the water absorption properties of the hydrophilic polymer. The matrix polymer described may include: polypropylene, polyethylene, polystyrene, ABS, SAN, polybutylene terephthalate, polyethylene terephthalate, nylon 6, nylon 6.6, nylon 4.6, nylon 12, phenolic resins, urea resins, epoxy resins, polyethylene vinyl acetate, polyethylene ethyl acrylate, polylactic acid, polysaccharides, polytetrafluoroethylene, polyimides, and polysulfones. The antimicrobial metal or metal ion is selected from the following: silver, copper, zinc, tin, gold, mercury, lead, iron, cobalt, nickel, manganese, arsenic, antimony, bismuth, barium, cadmium, chromium, thallium, and combinations thereof. The prior art differs from the present invention by way of antimicrobial metal ion release. The release of metal ions from the imbedded antimicrobial zeolites within the substrate of the current invention relies upon an ion exchange mechanism; versus the release mechanism described in the prior art where the release of the metal or metal-ions depends upon absorption of water. The technology of the prior art is intended for medical devices such as catheters but does not suggest the use of blood pressure cuff protective devices. Within the prior art, there is no mention of the use of melt-blown non-woven fabric, nor does it teach the method of which is used to manufacture the non-woven fabric.

U.S. Patent Publication No. 20050124724, assigned to 3M, illustrates a polymer composition comprising a hydrophilic polymer and a bioactive agent selected from the group consisting of a metal oxide of silver, copper, zinc, and combinations thereof; wherein the bioactive agent is dispersed within the hydrophilic polymer, and wherein substantially all of the bioactive agent has a particle size of less than one micron. The polymer composition can be prepared by a method combining components comprising a hydrophilic polymer, a metal compound from selected group consisting of a silver compound, a copper compound, a zinc compound, and combinations thereof. A hydroxide source converts the metal compound to the corresponding metal oxide, where in the components are combined in a manner to disperse the metal oxide within the hydrophilic polymer. The hydrophilic polymer is a carboxylic acid-containing organic polymer. The current invention differs from the prior art in that a silver and copper zeolite is the preferred antimicrobial agent to incorporate into the preferred thermoplastic polylactic acid (PLA) polymer melt-blown non-woven fabric.

U.S. Patent Publication No. 20040214495, assigned to Foss Manufacturing, describes a product including one or more component sections of thermoplastic polymer with incorporated anti-microbial additive with efficient sizing, placement, and quantity therein and at least one other component acting to afford a primary characteristic of one or more of strength, color, fire retardancy, odor suppression or modification, hydrophilic or hydrophobic characteristic promoting or suppressing, texture controlling and ultraviolet resistance to the product. The product as a whole is constructed and arranged to suppress microbial growth and/or to impart suppression action to an environment in which the product is ultimately used. The product comprises at least one indefinite form selected from the group consisting of yarn, tow, flat sheet, shaped sheet, film, monofilament, fabric, fabric laminate, film, film laminate, sheet, and fabric/film laminate. The product comprises a fabric section selected from the forms consisting of woven, knit, spun, non-woven (including fleece, air laid, flocked, needle punched spunbonded, spun laced and thermo bonded forms). The additive within the prior art is one selected from the group consisting of copper, zinc, tin, and silver. The prior art differs from the current invention by way of intended applications for the antimicrobial fiber. The prior art describes uses such as feminine hygiene products, diapers, wound dressings, burn dressings, etc. but does not suggest the use for blood pressure cuff protective devices. Further, the prior art does not describe the use of a melt-blown non-woven process to generate non-woven PLA fabric with imbedded antimicrobials. There is no mention of extent of antimicrobial efficacy for the prior art. Finally, not mentioned within the prior art is the lamination for support and fluid impermeability of the non-woven or woven fabrics with an adhesive and barrier film, which is taught within the current invention.

### BRIEF SUMMARY OF THE INVENTION

The above detailed examples of prior blood pressure cuff protective device inventions are inclusive of hygienic barriers meant to be applied to the appendage of the patient, as well as liners and covers designed to be incorporated with the blood pressure cuff itself. The present invention describes an advanced blood pressure cuff shield that attaches to the blood pressure cuff with the added value of antimicrobial efficacy, biodegradability, and multi-patient application.

The present invention encompasses a disposable and reusable barrier applied to the blood pressure cuff. In a preferred embodiment, the shield comprises a thin and flexible rectangular sheet with strategically placed adhesive strips in order to secure the shield to the blood pressure cuff without impeding the function of the hook and loop fastening system of the cuff itself. The sheet comprises at least one layer of non-woven material inclusive of antimicrobial agents and one non-porous, fluid impermeable layer. The non-woven material is oriented for skin contact, while the fluid impermeable barrier layer intimately contacts the pressure cuff. Fluid impermeability of the shield is preferable for the prevention of body fluids soiling or contaminating the blood pressure cuff. The adhesive strips run half the length of the shield, and are positioned to secure to the bladder end of the pressure cuff. The latter half remains unsecured as a “tail” to prevent interference of the fastening system of the blood pressure cuff.

The antimicrobial technology of the blood pressure cuff shield is a core aspect of this invention. The silver and copper zeolite imbedded biodegradable thermoplastic fibers act as a highly efficacious broad spectrum biocide, eradicating and inhibiting pathogenic microorganisms. This technology has proved to be highly effective against the more robust health care acquired infections including, but not limited to, MRSa, VRE, and Klebsiella pneumoniae.

The primary role of the present invention is to offer health care facilities a highly economic, multi-patient, antimicrobial, disposable, biodegradable, and robust blood pressure cuff shield allowing for 24 hours of protection for the device. These advantageous features, among others, documented below will become most apparent to those skilled in the art through the detailed description, figures, and claims.

### BRIEF DESCRIPTION OF THE FIGURES

FIG. 1 shows a schematic diagram of the embodiment of the blood pressure cuff shield.

FIG. 2 shows a schematic diagram of an embodiment of the production method of the present invention.

FIG. 3 shows a schematic diagram representing a side view of the blood pressure cuff shield attached to the blood pressure cuff, demonstrating the detached tail.

FIG. 4 shows a diagram of an embodiment with excess width to the present invention.

FIG. 5 shows a schematic diagram of an embodiment of the production method of the present invention.

FIG. 6 shows a schematic diagram of an embodiment of the calendering method of the present invention.

FIG. 7 shows a close-up photograph of the polylactic acid polymer meltblown non-woven fiber of the present invention.

FIG. 8 shows the antimicrobial efficacy of the non-woven with imbedded antimicrobial agents of the present invention.

FIG. 9 shows a demonstration of use of an embodiment of the present invention.

FIG. 10 shows a demonstration of use of the tensile grips for a 180 degree peel test.

FIG. 11 shows a photograph demonstrating that no residue from the adhesive remains on the blood pressure cuff upon removal.

FIG. 12 shows a photograph demonstrating the Biovation crafted conformability apparatus.

### DETAILED DESCRIPTION OF THE PREFERRED EMBODIMENTS

As used herein, the term “polymer” refers to thermoplastic, natural, naturally-derived, or synthetic, biopolymers and oligomeric species thereof. As used herein, the term “oligomer” refers to a low molecular weight polymer of two or more repeating monomeric repeating units. Polymers specifically include, but are not limited to, PolyLactic Acid (PLA); PolyCaproLactone (PCL) and PolyHydroxyAlkanoate (PHA) alone or in blends/alloys or as copolymers. As used herein, the term “shield” refers to a hygienic barrier that acts as a liner to the blood pressure cuff. As used herein, the term “tail” refers to the unfastened portion of the shield that remains free from the blood pressure cuff.

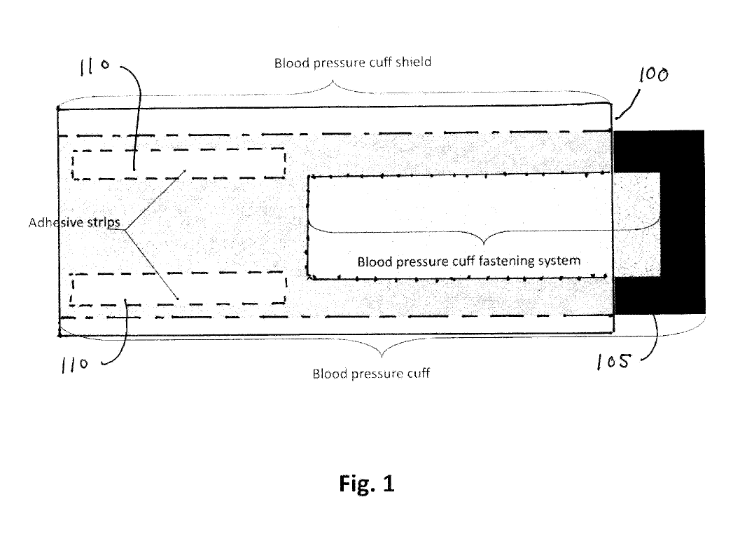

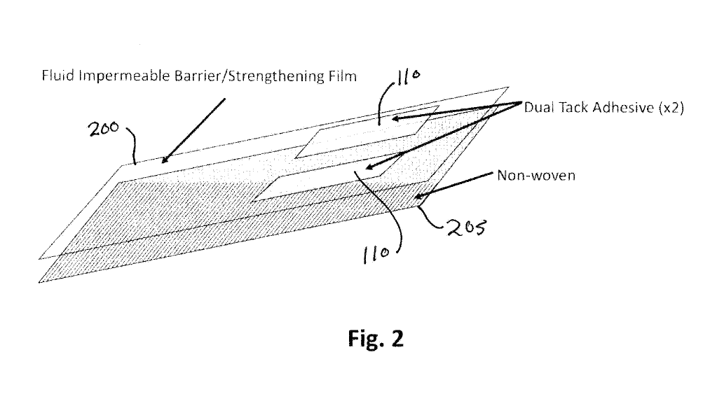

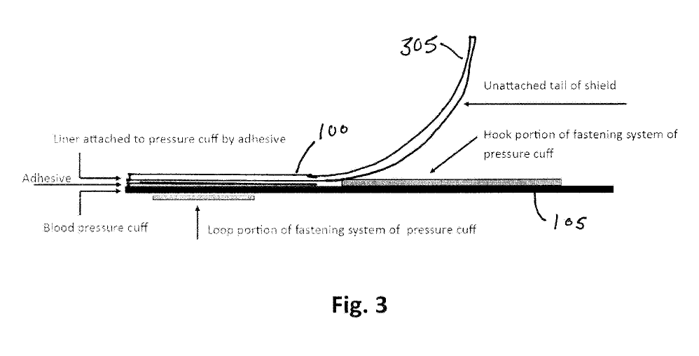

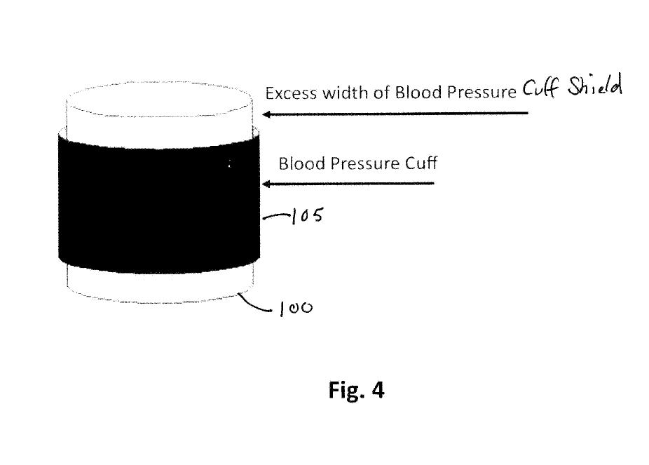

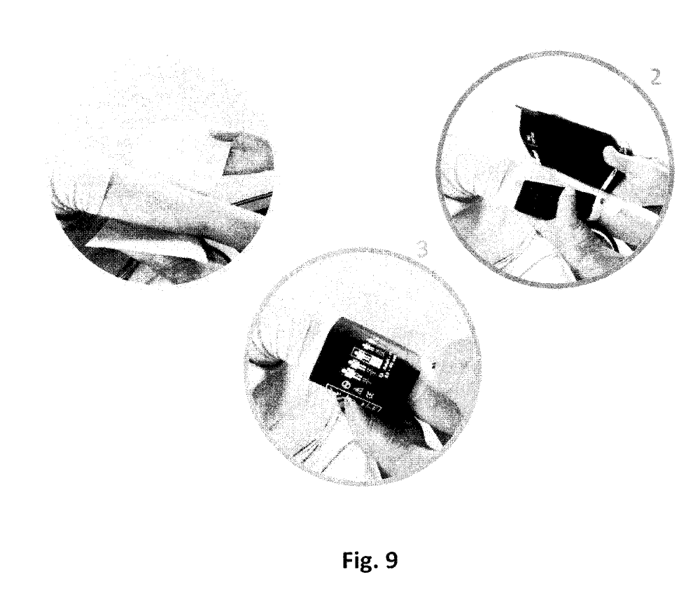

Adverting to the figures provided, the blood pressure cuff shield is depicted as covering almost the entirety of the blood pressure cuff shield, as shown in FIG. 1. The shield is preferably rectangular in shape and the width extends past the blood pressure cuff to ensure that there is buffer in circumstances where the shield is applied to the cuff slightly off center, as shown in FIG. 4. To accommodate the various sizes of blood pressure cuffs on the market, the present invention may be manufactured in numerous lengths and widths. The length of the blood pressure cuff shield should exceed the maximum of the arm circumference of the blood pressure cuff to ensure proper coverage. The shield is constructed from at least one layer of non-woven fabric, preferably PLA, but may be composed of a variety of melt-blown polymers, laminated to a fluid impermeable, structure strengthening barrier film. A fastening system, preferably dual tack adhesive strips, is then applied to the barrier film, as presented in FIG. 2. Though it is shown in the exemplary figure that two adhesives strips are used, the invention is not limited to this method or configuration. A larger area of adhesive may be used, and/or strips may be oriented differently, the primary function of the chosen fastening system is to secure the shield to the bladder portion of the blood pressure cuff. The adhesive fastening system is strategically positioned to avoid interference with the hook and loop fastening system of the blood pressure cuff itself. FIG. 3 displays a side view of the shield attached to the blood pressure cuff where it is apparent that the tail of the shield remains detached from the cuff. The innovative design of the tail allows the blood pressure cuff fastening system to be utilized as designed, while the addition of a replacement fastening system is not needed on the shield. In applying the blood pressure cuff to the patient, as demonstrated in FIG. 9, the bladder of the blood pressure cuff with attached liner is supported by the health-care provider in proper position against the patient's arm. Using the opposite hand, the tail of the liner is wrapped around the patient's arm and secured in place under the bladder end of the blood pressure cuff. The blood pressure cuff is then wrapped around the shield arm of the patient and secured via the hook and loop fastening system. This design is significantly different than the prior art.

The non-woven material layer prepared according to embodiments of the invention described herein utilizes natural or naturally-derived fibers, preferably polylactic acid, as the basis of the material for the sachet structure. The non-woven material is completely biodegradable; its composition can be varied to provide the ability to vary the degradation. The non-woven layer can also be modified with hydrophilic and hydrophobic materials to vary its ability absorb or repel bodily fluids. Antimicrobials can be incorporated into the non-woven fibers in a variety of ways (i.e. masterbatch and coating).

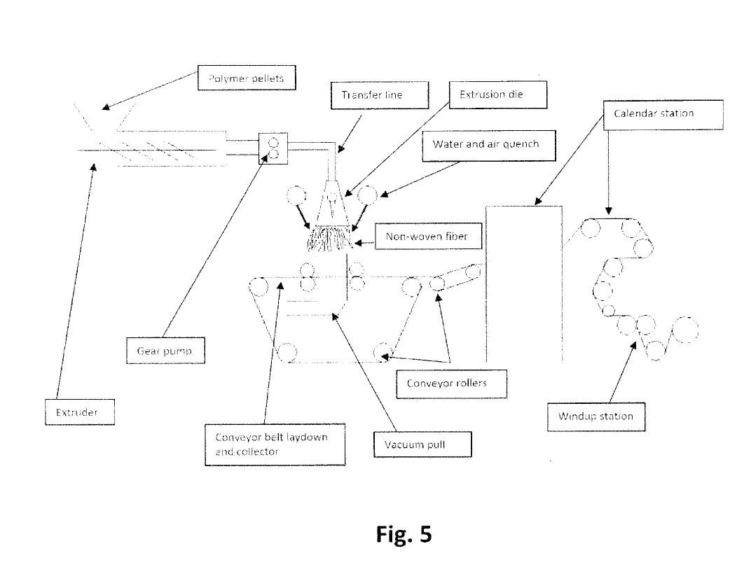

The methods described herein pertain to the creation of the non-woven fabric used for the skin contact portion of the blood pressure cuff shield. These are only exemplary and one of skill in the art will understand that, based on the teachings provided herein, modifications of these procedures are within the metes and bounds of the present invention. FIG. 5 shows a generic schematic of a meltblown system of which is used in the manufacturing of the non-woven goods.

NatureWorks (Minnetonka, Minn.) produces several grades of PLA in pellet form that can be melt processed into film or fibers and are useful in this invention. Many grades are useful however grade 6202D as a high melt-point version with the optional use of grade 6251 D as a low-melt binder fiber have proven to process well in the present invention. Perstorp (Toledo, Ohio) produces PCL and, although several grades are suitable for use in the present invention, grade Capa 6800 processes well. Mirel PHA from Metabolix (Cambridge, Mass.) is also compatible with the present invention.

When processing PLA, to maintain maximum chain length, it is important to dry the polymer in a commercial desiccant dryer such as a Conair (Cranberry Township, Pa.) “W” series machine to a moisture level below 200 ppm. This is critical as PLA polymer is extremely hygroscopic and will acquire moisture from the air rapidly. This moisture hydrolytically degrades the polymer chains resulting in a reduced viscosity and thus product strength. If moisture levels are too high, the additional problem of steam generation and uncontrolled pressures within the extrusion system are observed.

For exemplification, for production, a Davis-Standard (Pawcatuck, Conn.) single screw 30:1 2.5″ extruder (or equivalent) with melt temperatures of 350 to 425° F. and pressures of 500 to 2000 psi are achieved at the outlet. The polymer passes through filtration to remove particulate debris and enters a pressure control zone achieved via a positive displacement Zenith (Monroe, N.C.) gear pump. Molten pressurized polymer is delivered to a melt-spinning die produced by BIAX (Greenville, Wis.). Several arrangements of nozzles, diameters, and total nozzle count can be varied to suit the polymer and final production needs. A typical spinning die contains 4000-8000 nozzles/meter of width with an internal diameter of 0.25-0.50 mm may be utilized efficiently. It must be noted that melt spinning dies produced by other suppliers such as Hills (W. Melbourne, Fla.) or Reifenhauser (Danvers, Mass.) may be used.

Heated and high velocity air is introduced into the die and both polymer and air streams are released in close proximity allowing the air to attenuate the polymer streams as they exit the die. Air temperatures of about 230-290° C. with pressures at the die at about 0.6 to about 4.0 atmospheres may be used. Following extrusion and attenuation, cool and/or moist air may be used to quench the fibers rapidly. At this point, liquids or mists can be applied to coat the surface. Surfactants, antimicrobials, or adhesives can be beneficially adhered to the fibers.

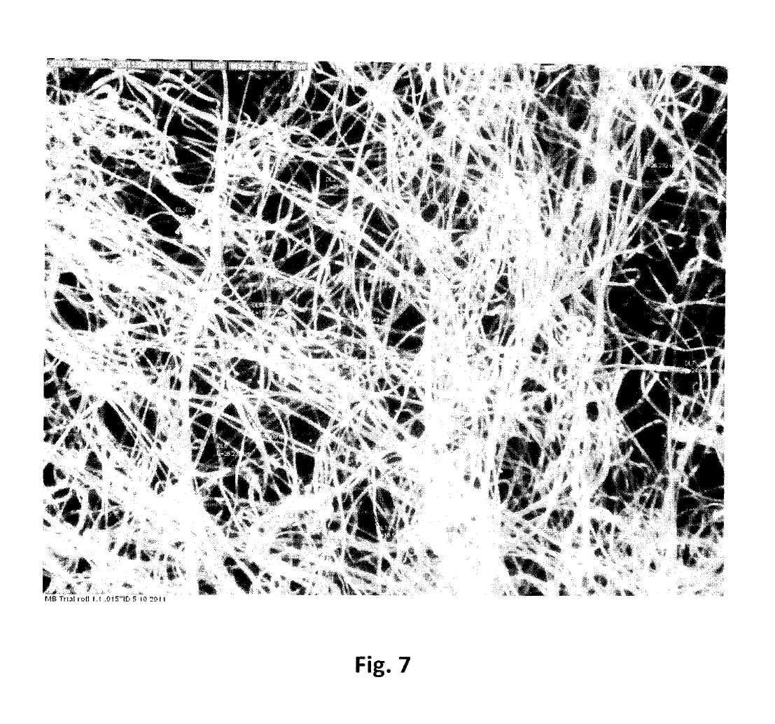

The fibers may be collected on a single belt or drum or a multiple belt or drum collector. Air is drawn from below the belt(s) or drum(s) and fibers collect in a web or matt on the surface, as demonstrated in FIG. 7. There are many adjustments in the entire system, temperatures, pressures, quench conditions, extrusion air velocity, suction air velocity, etc. With these adjustment points, a matt that is, for example, stiff and thin or flexible and fluffy is possible. For this invention, a low-density structure with fine-diameter fibers is beneficial although one of skill in the art will realize that other densities and diameters are suitable for use in the present invention.

Fiber diameters can range from approximately 1 to 30 microns (μm), however it is possible to produce nano or sub-micron fibers via increased hot air attenuation and/or low polymer throughputs. The cost of production increases as a result however, the overall surface area of the fibers increases. Likewise, larger fibers are easily produced when attenuation air is reduced or eliminated and/or melt pressures are increased. A compromise of cost and performance is seen in, approximately, the 5-25 micron range. Within the large number of consecutive fibers being spun, it can be important to allow a range of diameters as this has been observed to increase the loft or thickness of the structure and this provides for improved shock absorbing and cushioning properties. Different diameters can be achieved by adjusting the internal nozzle diameters and/or air velocity at certain nozzles or by directing external cooling air toward certain fiber streams.

It is preferred to place antimicrobial agents in the polymer (as described and exemplified throughout the present specification) and, thus, in each fiber and/or interspersed between fibers. This invention utilizes, but is not limited to, antimicrobial action generated in situ upon contact of the pathogen with the antimicrobial agent. The antimicrobial is inclusive of silver and copper containing zeolite (Aglon, Wakefield, Mass.), which is imbedded within the biodegradable non-woven, thermoplastic polymer fibers that comprise the substrate that comes in contact with the patients' skin. The zeolite impregnated non-woven fiber destroys or prevents microbial growth on the blood pressure cuff shield. Such biodegradable and low bioburden fibers include those based on poly(lactic) acid, also known as polylactide, and its various L, D and meso configurations, including mixed L, D, and meso compositions, their various crystallinities, molecular weights, and various co-polymers. In this work polylactic acid is understood to be synonymous with polylactide and both terms encompass all the optically active variations of the polymer. Other examples of antimicrobial, low bioburden polymers are known to those in the art, e.g., as shown in a review by Kenway, et. al., (Kenway, E. R., Worley, S. D., Broughton, R. (2007). The chemistry and applications of antimicrobial polymers—A state-of-the-art review; Biomacromolecules, 8 vol, number 5 1359-1384).

A preferred antimicrobial agent is ionic silver, being released from a nonwoven material made preferably from polylactic acid fibers. Examples of suitable silver and silver ion-based agents include, but are not limited to, silver halides, nitrates, nitrites, selenites, selenides, sulphites, sulphates, sulphadiazine, silver polysaccharides where such polysaccharides include simple sugars to polymeric and fibrous polysaccharides, silver zirconium complexes, forms including organic-silver complexes such as silver trapped in or by synthetic, natural or naturally-derived polymers, including cyclodextrins; all compounds, inorganic or organic, that contain silver as part of the structure, where such structures can exist as a gas, solid, or liquid, as intact salts, dissolved salts, dissociated species in protic or aprotic solvents and silver species which contain the molecular morphology or macroscopic properties of materials in contact with silver whereby such materials, either organic, inorganic, and/or of biological nature, are found in various morphologies, such as crystalline or amorphous forms, or optical activities, such as d, I or meso forms, or tacticities such as isotactic, atactic, or syndiotactic, or mixtures thereof of any of the above.

Another preferred antimicrobial agent is ionic copper, being released from a nonwoven material made preferably from polylactic acid fibers. Examples of suitable copper and copper ion-based agents include, but are not limited to, copper halides, acetates, carbonate, nitrates, nitrites, selenites, selenides, sulphides, sulphates, sulphadiazine, copper polysaccharides where such polysaccharides include simple sugars to polymeric and fibrous polysaccharides, copper zirconium complexes, or copper complexes thereof.

Silver and copper ion-based agents include, for example, compounds that contain silver or copper as part of the structure that can be covalently bound, ionically bound, or bound by other mechanisms known as “charge-transfer” complexes, including clathrate compounds that involve silver, silver species, copper, or copper species as part of the structure. Silver and copper ion-based agents also include silver, silver containing species, copper, or copper containing species that exist as a result of the process of sorption, either chemical or physical sorption, meaning absorption or adsorption, where the sorptive surface can be a molecule, polymer, organic or inorganic entity such as, but not limited to, synthetic oligomers or polymers (either thermoplastic or thermoforming), natural or naturally-derived polymers (either thermoplastic or thermoforming), biodegradable and non-biodegradable polymers (either thermoplastic or thermoforming), and inorganic or organic species whose surface area provides for some sorptive effect including, but not limited to, charcoal, zeolites of all chemical structures, silica, diatoms, and other high-surface area materials, also including silver or silver species in all its known valence states, either organically or inorganically bound, and includes organic or inorganic materials, either gas, liquid, or solid, where the silver or silver species can “exchange” or transfer by mechanisms such as, but not limited to, ion-exchange, diffusion, replacement, dissolution, and the like, including silver glass, silver zeolite, silver and copper zeolite, copper zeolite, silver-acrlyic and nano-silver structures. Zeolite carrier based (the silver and copper ions exchange with other positive ions (often sodium) from the moisture in the environment, effecting a release of silver and copper “on demand” from the zeolite crystals) and glass based silver chemistries (soluble glass containing antimicrobial metal ions wherein with the presence of water or moisture, the glass will release the metal ions gradually to function as antimicrobial agents), are non-limiting examples of copper and silver-ion-based agents suitable for use in the present invention.

Any combination of the above exemplary silver, silver ion-based, copper ion-based and copper agents is also contemplated for use in the blood pressure cuff shield of the present invention.

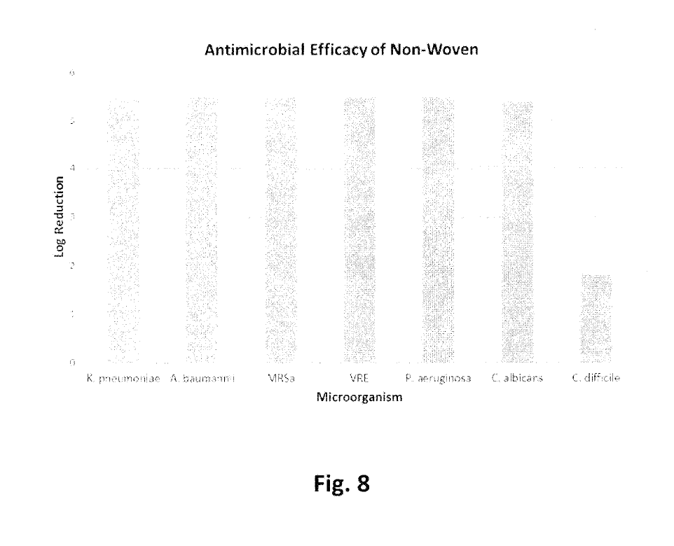

In a preferred embodiment of the present invention, the antimicrobial and antifungal agents are incorporated into the actual fibers of the skin contact non-woven material of the blood pressure cuff shield. In this embodiment, the agents are added to the polymer prior to the formation of the polymer into fibers. The antimicrobial and antifungal agents are interspersed between the fibers of the non-woven material. In this embodiment, the antimicrobial and antifungal agents are both incorporated into the actual fibers. FIG. 8 demonstrates the broad spectrum antimicrobial efficacy, of the preferred embodiment, against VRE, MRSa, Klebsiella pneumoniae, A. baumannii, P. aeruginosa, C. albicans, and C. difficile.

In other embodiments, non-silver and non-silver ion-based antimicrobial and antifungal agents are contemplated for use with the blood pressure cuff shield of the present invention. These non-silver and non-silver ion-based agents may be used in conjunction with the silver and silver ion-based agents of the present invention. One of ordinary skill in the art, based on the teachings of the present specification, can determine suitable combinations of agents depending on the fiber composition of the blood pressure cuff shield. Suitable non-silver and non-silver ion-based agents are, but are not limited to, compounds containing zinc, copper, titanium, magnesium, quaternary ammonium, silane (alkyltrialkoxysilanes) quaternary ammonium cadmium, mercury, biguanides, amines, glucoprotamine, chitosan, trichlocarban, triclosan (diphenyl ether (bis-phenyl) derivative known as either 2,4,4′-trichloro-2′ hydroxy dipenyl ether or 5-chloro-2-(2,4-dichloro phenoxyl) phenol), aldehydes, halogens, isothiazones, peroxo compounds, n-halamines, cyclodextrins, nanoparticles of noble metals and metal oxides, chloroxynol, tributyltins, triphenyltins, fluconazole, nystatin, amphotericin B, chlorohexidine, alkylated polethylenimine, lactoferrin, tetracycline, gatifloxacin, sodium hypophosphite monohydrate, sodium hypochlorite, phenolic, glutaraldehyde, hypochlorite, ortho-phthalaldehyde, peracetic acid, chlorhexidine gluconate, hexachlorophene, alcohols, iodophores, acetic acid, citric acid, lactic acid, allyl isothiocyanate, alkylresorcinols, pyrimethanil, potassium sorbate, pectin, nisin, lauryl arginate, cumin oil, oregano oil, pimento oil, tartaric acid, thyme oil, garlic oil (composed of sulfur compounds such as allicin, diallyl disulfide and diallyl trisulfide), grapefruit seed extract, ascorbic acid, sorbic acid, calcium compounds, phytoalexins, methyl paraben, sodium benzoate, linalool, methyl chavicol, lysozyme, ethylenediamine tetracetic acid, pediocin, sodium lactate, phytic acid, benzoic anhydride, carvacrol, eugenol, geraniol, terpineol, thymol, imazalil, lauric acid, palmitoleic acid, phenolic compounds, propionic acid, sorbic acid anhydride, propyl paraben, sorbic acid harpin-protein, ipradion, 1-methylcyclopropene, polygalacturonase, benzoic acid, hexanal, 1-hexanol, 2-hexen-1-ol, 6-nonenal, 3-nonen-2-one, methyl salicylate, sodium bicarbonate and potassium dioxide.

Thus, in an embodiment of the present invention, the invention comprises an antimicrobial, biodegradable blood pressure cuff shield, comprising at least one layer of non-woven fibers comprising one or more biodegradable thermoplastic polymers and one or more silver-based, silver ion-based, copper-based, non-silver based, and/or non-copper based antimicrobial agents.

In our current invention, although we can utilize synthetic fibers such as polypropylene and polyethylene, or paper such as recycled paper, we preferentially employ natural plant-based materials, such as natural polymers or naturally-derived meltblown nonwoven polymer fibers or filaments. One example is polylactic acid (PLA), as defined above. The PLA is degradable and renewable, and has a low bioburden as opposed to, for example, recycled wood pulp. From an end-use standpoint and a processing and manufacturing standpoint, the low bioburden profile achieved with the nonwoven process precludes any heat drying that is required to destroy microbes present in a wood or tissue-based product; allowing a “cleaner” and safer system when compared to traditional alternatives such as wood pulp.

Another differentiating feature of PLA is that PLA is completely compostable, resorbable and safe in terms of cytotoxity, versus recycled pulp or synthetic fibers. One of the degradation products of polylactic acid is lactic acid, which is produced in the human body.

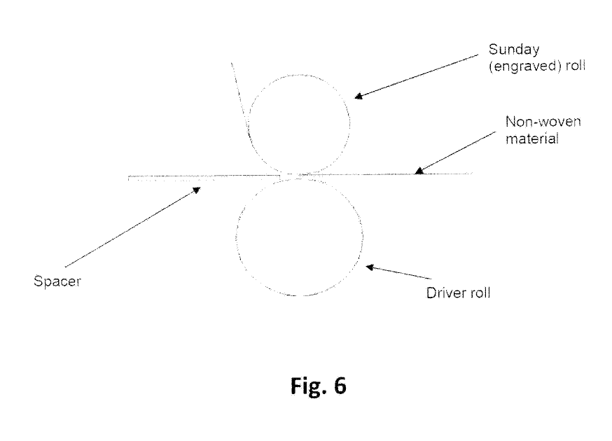

In our invention the PLA can be thermally glazed (also known as “calendering”). FIG. 6 provides a general schematic of the system used for calendering. This is a distinct advantage over conventional materials. Heat with calendering and even exposure to blasts of hot air can render the nonwoven filaments with a smooth film-like surface, yet still have porosity to fluids and moisture. With regard to the present invention, the calendering process and the effect it has on the surface of the non-woven thermoplastic skin contact layer. Porosity can be controlled by controlling the heat used to calendaer the material, and by the usage of an engraving roll that can place apertures on the film. Glazing can be an overall surface treatment or a variable/zone application. For purposes of visual comparison only, and not for comparison to mechanical or end-use properties, the smooth glazed PLA fibrous surface resembles in looks only the commercial product Tyvek®. The purpose of the fiber glazing (calendering) process is to eliminate the need for a separate film, and it provides a unique and advantageous method to control bodily fluid with a minimum of lamination and processing effort while increasing the utility of the blood pressure cuff shield. One of ordinary skill in the art would be able, with guidance from the teachings of the present invention, to extrapolate times and temperatures necessary for a desired porosity.

In a further embodiment of the present invention, antibacterial agents can be added into the polymer that is then meltblown into fibers. In other words, the antimicrobial agents are incorporated into the polymer fibers of the present invention. This provides protection and encapsulation of the antimicrobial agents and provides fast acting protection against patient communicated pathogens. Antibacterial, antimicrobial and antifungal agents can also be incorporated into the non-woven material of the present invention in a variety of ways.

In an embodiment of the present invention, the antimicrobial action is incorporated into the polymer fiber structure of the present invention. The presence of the antimicrobial agent(s) in the non-woven material eradicates pathogens on contact with the shield. It also prohibits the spread of pathogens from the patient to the blood pressure cuff, which would nominally acquire a cocktail of microorganisms during use.

One improvement of the present invention over the related prior art is that the present invention integrates the antimicrobial compound as a masterbatch directly into the thermoplastic (e.g., polylactic acid) fibers as part of the meltblown fiber manufacturing process with specifically tuned process variables (as exemplified below) which results in the non-woven material used as the skin contact material of the blood pressure cuff shield. Additionally, an improvement of the present invention is to be able to specifically calender (as a function of speed, pressure and temperature) the polylactic acid polymer non-woven material with the antimicrobial formulation in order to allow it to function as a fluid barrier and/or to impart a soft/smooth feel for patient comfort.

One novel and unique improvement of the present invention over the related prior art is the construction of the blood pressure cuff shield from polylactic acid in a novel fashion that allows multiple layers of non-woven polylactic acid fibers to manufactured and calendered allowing flexibility and optimization while ensuring the robustness of the non-woven material layer(s) in order it to function as a reusable blood pressure cuff shield while imparting comfort for the patient.

For additional support and fluid impermeability, a barrier film is laminated to the non-woven fabric by way of hot melt, pressure sensitive acrylic adhesive, silicone adhesives, and poly-urethane adhesives, but the lamination of the barrier film is not limited to the preceding methods. The flexible barrier film may be composed of a 1-3 mil biaxially oriented poly propylene, compostable polystyrene, thermoplastic polyurethane, thermoplastic polyolephin blends, polyamides, or any type of thermoplastic copolymer thereof, most preferably a thermoplastic co-polyester. The blood pressure cuff shield is designed to withstand a great deal of stress and strain imparted by the multiple inflations and deflations throughout the intended 24 hour period of use. The barrier film lends the non-woven material the necessary durability to tolerate the repetitive tension through the cycle of use. Lastly, the barrier film prevents penetration of bodily fluids of which could soil and/or contaminate the blood pressure cuff.

To secure the blood pressure cuff shield to the blood pressure cuff, a fastening system is incorporated with the shield. The placement of the fastening system of the shield is important in order to avoid interferring with the fastening system of the blood pressure cuff. In order to secure the blood pressure cuff shield to the blood pressure cuff, the shield is attached to the bladder end of the cuff as demonstrated in FIG. 3. The fastening system of the shield may comprise buttons, hook and loop, or most preferably adhesive. A variety of adhesives can be used including silicone, polyurethane, and pressure sensitive adhesives. The adhesive can be arranged in strips, as a block covering the entirety of the bladder end, or as a pattern. The chosen adhesive will allow the shield to be removed from the blood pressure cuff with no remaining residue on the cuff.

### EXEMPLIFICATION

### Example 1

### Creation of the PLA Non-Woven Blood Pressure Cuff Shield Material

Grade 6202D PLA polymer pellets from NatureWorks (Minnetonka, Minn.) were utilized from a fresh unopened bag and introduced into the mouth of a 2.5″ 30:1 40-hp extruder and exposed to mechanical shear and heat ranging from approximately 350 to 450° F. as it travels through the system. Filtration followed by a gear pump pushed the molten polymer through a heated transfer line into a BIAX meltblown system at approximately 800 to 1500 psi. Compressed air was heated to approximately 475-525° F., introduced into the die at approximately 10-18 psi and used to attenuate the PLA fibers through nozzles with an internal diameter of about 0.012 inches. A filtered water mist quench was produced using a high-pressure piston pump and a fluid-misting system. This quench was operated at approximately 500-1800 psi and the mist impinges the fibers as they exit the die zone which serves to cool them. An air quench system introduced cool outside air to the fibers before they were deposited on a flat belt with a vacuum source below. The speed of this belt determined the weight of the web. For the blood pressure cuff, a weight of between 80 to 100 grams per square meter (gsm) is required. The vacuum level additionally served to compress the web, or allow it to remain fluffy and at a low density. Calender bonding served to strengthen and smooth the non-woven web.

To calender bond the non-woven film, we utilized a BF Perkins (division of Standex Engraving, LLC, Sandston, Va.) Calender Station which contained two heated rolls and two hydraulic rams. Each heated roll was filled with high temperature oil, which was heated by a separate machine. A hot oil machine controlled the temperature and the flow of oil through each zone of the Calender Station. The temperature can range from 110 to 550° F. The hot oil was circulated at 30 psi through 2 inch iron pipes into a rotary valve for each zone.

The Calender Station was opened and closed by a control station which also regulated the amount of pressure used to move the hydraulic rams. This pressure can range from 1 psi to 3,000 psi and maintained the amount of force with which the Drive Roll was supported. A variable spacer between the Sunday Roll (also called an Engraved Roll) and the Drive Roll maintained the distance of one roll to the other. The spacer allowed for the thickness of the PLA and the hydraulic rams maintain that distance. See FIG. 2 for a schematic representation of the process. Non-limiting specifications are given below. One of ordinary skill in the art will be able to modify these specifications based on the guidance provided by this specification.
i. Top roll, labeled Sunday Roll, was a smooth roll; 10″ diameter by 19½″ length.
ii. Bottom Roll, labeled Drive Roll, was a smooth roll; 10″ diameter by 19½″ length.
iii. The temperature was variable on product density and speed of the process line. The speed can range, for example, from 1 to 200 FPM (feet per minute) with a temperature of 175 to 350° F.
iv. The distance between the rolls was a variable controlling product thickness which can range from 0.5 to 0.001 inch.

Different variations of PLA calendered film, inclusive of apertures, can be manufactured with different mechanical properties based on the teachings of the present specification. For example, PLA Film 1 was calendered 33 gsm PLA integrated with a formulation of silver Zeolite grade AC-10D from AgION coupled with silver glass grade WPA from Marubeni/Ishizuka at 240° F., 40 fpm, at 0.001 inch gap under 900 psi. PLA Film 2 was calendered 66 gsm melt spun PLA integrated with a formulation of silver Zeolite grade AC-10D from AgION coupled with silver glass grade WPA from Marubeni/Ishizuka at 280° F., at 10 fpm, at 0.005 inch gap, under 1,000 psi. Corresponding test data is shown below in Table 1, below.

Table 1

If the corresponding PLA Film 1 and PLA Film 2 were uncalendered, the data is as follows (which clearly shows the effects of calendering):

g/hm²=grams per hour times meter squared

TABLE 1

| | | | Permeation |
|---|---|---|---|
| | Tensile Strength | Apparent | (ASTM E96) |
| | (ASTM D5030) | elongation (%) | (g/hm²) |
| PLA Film 1 | 2.999 in/lbs | 6.884% | 80.2337 |
| PLA Film 2 | 5.579 in/lbs | 5.064% | 67.7960 |
| PLA Film 1 - | 0.765 in/lbs | 5.886% | 67.4622 |
| uncalendered | | | |
| PLA Film 2 - | 3.784 in/lbs | 3.814% | 64.9974 |
| uncalendered | | | |

Once the non-woven was calendered it was directed to a windup station for final packaging and assembly. Refer to FIG. 1 for a schematic view of the process.

### Example 2

### Non-Woven Fiber Material Made with Polypropylene Resin

This is similar to the above example with the exception of polypropylene polymer (PP) is substituted for the PLA. The advantage of PP is a higher processing and throughput speed. PP has all the required health and safety and low-bioburden properties medical dressings require. It is also receptive to hydrophilic additives in a masterbatch or surface treatment to impart rapid fluid wet-out. Additives can easily be included in masterbatch form. A PP meltblown web can also be thermally point bonded or placed on a spunbond carrier for additional strength and can be processed in a secondary treatment step to impart a silver-containing treatment.

In this example we used Exxon Mobil (Houston, Tex.) Achieve 6936G ultra-high melt flow rate polypropylene at the 100% level and with additives. One distinct advantage was lower melt processing conditions when compared to PLA. Extruder and spinning temperatures in the 275 to 350° F. range were sufficient and this product and this allowed polymer additives that were heat-intolerant to be utilized. Melt spun PP of various densities and thicknesses were calendered at a close nip under high pressure to produce a film structure. See test data below (Table 16) to see the various structures created and the performance difference between “calendered” and “uncalendered.”

The 33 gsm melt spun PP was calendered at 210° F., at 10 fpm (feet per minute), at 0.001″ gap, under 1000 psi, to create “PP Film 1”; see Table 2, below.

TABLE 2

| | Tensile Strength | Apparent Elongation |
|---|---|---|
| | (ASTM D5035), in/lbs | (%) |
| PP Film 1 - Un-Calendered | 1.253 | 29.30 |
| PP Film 1 - Calendered | 2.294 | 15.78 |

A 48 gsm melt spun PP was calendered at 250° F., at 10 fpm, at 0.005″ gap, under 1,000 psi, to create “PP Film 2,” see, Table 3, below.

TABLE 3

| | Tensile Strength | Apparent Elongation |
|---|---|---|
| | (ASTM D5035), in/lbs | (%) |
| PP Film 2 - Un-Calendered | 1.788 | 23.398 |
| PP Film 2 - Calendered | 3.789 | 8.475 |

### Example 3

### Active Structure Made with Polycaprolactone Resin

This is similar to the above examples with the exception that Polycaprolactone (PCL) was added to the PLA in a blend at various levels from 5% to over 70%. PCL is a naturally derived polymer with a very low melt point. When used at low levels, generally 30% and lower, it functions as a plasticizer for the PLA, a brittle polymer, and imparts lubricity and softness to the fibers that functions to reduce breakage. This dramatic improvement is apparent even at a 2% add-on level and increases with concentration. The PLA/PCL blend can also incorporate masterbatch additives or surface finishes to control surface hydrophilicity and fluid wet-out. Silver can also be incorporated into the blended fibers as previously described. The lower processing temperature of the PCL allows the use of low-temp additives but also limits the effective storage and use temperatures of the finished product.

Below, Table 4 shows the physical and mechanical properties of various PLA/PCL structures that were manufactured. For example, PLA/PCL Structure UC-1 is non-calendered 600 gsm 93% PLA with 3% CP-L01 and 3% CT-L01 and 1% PCL run at 400° F., 3 fpm and 1100 psi. Corresponding test data is shown below for various combinations and permutations wherein the speed, pressure and temperature were changed.

TABLE 4

| | Tensile | | |
|---|---|---|---|
| | Strength | Apparent | |
| | (ASTM | elongation | Break Time |
| | D5035) | (%) | (sec) |
| PLA/PCL Structure UC1 | 0.732 | 28.996 | 4.375 |
| PLA/PCL Structure UC2 | 0.937 | 14.131 | 2.141 |
| PLA/PCL Structure UC3 | 1.109 | 16.356 | 2.547 |
| PLA/PCL Structure UC4 | 1.837 | 12.024 | 1.843 |
| PLA/PCL Structure UC5 | 1.731 | 21.465 | 3.313 |
| PLA/PCL Structure UC6 | 1.347 | 22.304 | 3.391 |
| PLA/PCL Structure UC7 | 1.840 | 23.915 | 3.609 |
| PLA/PCL Structure UC8 | 1.360 | 10.460 | 1.594 |
| PLA/PCL Structure UC9 | 1.375 | 18.804 | 2.844 |
| PLA/PCL Structure UC10 | 1.767 | 17.139 | 2.734 |
| PLA/PCL Structure UC11 | 1.730 | 25.954 | 4.000 |
| PLA/PCL Structure UC12 | 1.316 | 21.022 | 3.250 |
| PLA/PCL Structure UC13 | 0.797 | 22.914 | 3.469 |
| PLA/PCL Structure UC14 | 1.176 | 15.248 | 2.312 |
| PLA/PCL Structure UC15 | 0.755 | 27.581 | 4.157 |
| PLA/PCL Structure UC16 | 0.851 | 19.247 | 2.906 |
| PLA/PCL Structure UC17 | 1.205 | 20.022 | 3.094 |
| PLA/PCL Structure UC18 | 1.118 | 23.247 | 3.562 |

The mean is 1.277 lbs for tensile strength, 20.046% for apparent elongation and 3.063 sec for break time.

By calendering various samples, the following data shown in Table 5, below, was obtained:

TABLE 5

| | Tensile | | |
|---|---|---|---|
| | Strength | Apparent | |
| | (ASTM | elongation | Break Time |
| | D5035) | (%) | (sec) |
| PLA/PCL Structure 1 | 1.957 | 18.478 | 2.797 |
| PLA/PCL Structure 2 | 1.636 | 15.690 | 2.468 |
| PLA/PCL Structure 3 | 1.702 | 16.475 | 2.500 |
| PLA/PCL Structure 4 | 1.621 | 14.251 | 2.157 |
| PLA/PCL Structure 5 | 1.357 | 12.808 | 1.937 |
| PLA/PCL Structure 6 | 2.032 | 12.911 | 1.953 |
| PLA/PCL Structure 7 | 1.117 | 23.799 | 3.593 |
| PLA/PCL Structure 8 | 1.481 | 10.696 | 1.704 |
| PLA/PCL Structure 9 | 2.268 | 19.359 | 3.000 |
| PLA/PCL Structure 10 | 2.221 | 17.755 | 2.750 |
| PLA/PCL Structure 11 | 2.185 | 22.342 | 3.375 |

The mean is 1.780 lbs for tensile strength, 16.779% for apparent elongation and 2.567 sec for break time.

### Example 4

### Active Structure with Topical Hydrophilic Treatment Added for PLA

This is similar to Example 1 except the hydrophilic additive was in liquid form mixed into the water quench system and sprayed directly on the fibers while hot. Many surfactants are candidates; however polyethylene glycol (PEG) 200-900 molecular weight (mw) is preferred. The concentration used was based on the weight of the fibers strayed and a range of 0.05% to 2.0% has proved beneficial in promoting rapid fiber wet-out. Additionally, the resultant fibrous web demonstrates a more rapid fluid acquisition speed was observed. This enhanced hydrophilicity was advantageous when an absorbent article with rapid fluid uptake was desired. Another product, Triton X-100 (Dow Chemical, Midland, Mich.) was also tried successfully. It was applied to a 3×3 inch, 33 gsm PLA non-woven comprising a formulation of silver Zeolite grade AC-10D from AgION coupled with silver glass grade WPA from Marubeni/Ishizuka, with a water mixture, at 1% and 0.5%. Each sample was fully submerged into a volume of water and then weighed with these results (Table 6).

TABLE 6

| | Dry Weight (g) | Wet Weight (g) |
|---|---|---|
| 0% Triton X-100 | 0.19 | 0.45 |
| 0.5% Triton X-100 | 0.19 | 1.66 |
| 1% Triton X-100 | 0.19 | 1.72 |

Repeated insult performance is important to determine the robustness of the material. The above samples were re-tested for repeated insult performance by saturating and drying each sample five times to determine if the hydrophilic properties were consistent after multiple uses. The positive results are presented below (Table 7).

TABLE 7

| | Dry Weight after 5 | Wet Weight after |
|---|---|---|
| | insults (g) | 5 insults (g) |
| 0% Triton X-100 | 0.19 | 0.75 |
| 0.5% Triton X-100 | 0.19 | 1.86 |
| 1% Triton X-100 | 0.19 | 1.93 |

Similar results were obtained with polypropylene based on the guidance provided by the present specification for those of ordinary skill in the art.

A 33 gsm polypropylene material was created with 3% TMP12713, a modifier manufactured by Techmere (Clinton, Tenn.); a 3″ by 3″ sample was cut and submerged into a volume of water and then weighed. The sample was re-tested, saturated and dried multiple times with these results (Table 8):

TABLE 8

| | Dry Weight (g) | Wet Weight (g) |
|---|---|---|
| 1^{st} insult | 0.19 | 1.85 |
| 5^{th} insult | 0.19 | 1.94 |

### Example 5

### Incorporation of Ionic Silver and Copper Antimicrobial Properties

This example is similar to Exhibit 1 except a custom masterbatch containing a silver/copper ion compound was incorporated to provide broad antimicrobial and antifungal performance. Several silver-releasing materials have been evaluated including, silver/copper Zeolite grade AC-10D from AgION, silver glass grade WPA from Marubeni/Ishizuka, silver zirconium, AlphaSan from Milliken (Spartanburg, S.C.). In each case, a 20-30% loading in a carrier polymer (Dupont Elvaloy AC, Wilmington, Del.) was prepared and used to uniformly deliver the silver additive into the mix. One preferred silver agent was the silver/copper zeolite grade AC-10D from AgION which contained copper elements as an anti-fungal agent. Another preferred silver was the WPA silver glass powder from Marubeni/Ishizuka. Particle size of less-than 5 microns was specified with an average of 2-3 microns to preclude spinneret nozzle clogging. The final concentration of silver and copper in the meltblown fibers was dependent on the quantity of masterbatch used. In trials, up to 20% masterbatch has been processed to demonstrate an extreme loading, up to 5.0% silver and 7.0% by weight. A silver and copper loading of 2000-2500 ppm and 2500-3200 ppm, respectively, is required in order to achieve the required performance of the blood pressure cuff shield. Refer to Table 10 for antimicrobial efficacy data in Example 6. In this application, silver and copper were highly effective as long-term bacterial control properties match the end-use requirements. The silver and copper zeolite powder was compounded with the PLA polymer to create a masterbatch of the antimicrobial additive. PLA was chosen as the carrier polymer due to its biodegradable/compostable properties.

As a reference for mechanical properties, the tensile strength of one 100 gsm PLA layer was measured to be 5.549 in/lbs using a Thwing-Albert (West Berlin, N.J.) Tensile Tester using ASTM D5035 protocols (as is known to those of ordinary skill in the art).

### Example 6

### Active Structure with Polymer Additives for Lubrication of PLA

This example is similar to Example 1, above, however a polymer additive or masterbatch in dry form was added into the PLA to impart lubricity. When added to the PLA at a 3.0% level higher volumetric throughput rate was observed (higher density; i.e., gsm attainment) while maintaining the same operating pressures, indicating a lower resistance to pumping. The higher volumetric throughput rate was observed by the increased rpm on the melt-pump and extruder motor. The melt additive used was CP-L01 from Polyvel Inc. (Hammonton, N.J.), a multipurpose plasticizer additive. When CT-L01 was substituted, also from Polyvel, at 3% level, lubricant or processing aid for “slip,” the same throughput rate at lower extruder and melt-pump speeds was observed.

The data below (Table 9) shows the change in density (gsm) for different runs of PLA integrated with a formulation of silver Zeolite grade AC-10D from AgION coupled with silver glass grade WPA from Marubeni/Ishizuka with different process settings and with different levels of additives.

TABLE 9

| | Density, extruder speed (rpm) |
|---|---|
| | and Melt-pump speed (rpm) |
| PLA non-woven material | 63 gsm, Extruder RPM 12%, |
| | Melt Pump RPM 19% |
| 97% PLA with 3% CP-L01 non-woven | 65 gsm, Extruder RPM 13.5%, |
| material | Melt Pump RPM 21% |
| 97% PLA with 3% CT-L01 non-woven | 55 gsm, Extruder RPM 11%, |
| material | Melt Pump RPM 18% |
| 94% PLA with 3% CP-L01 and 3% CT- | 63 gsm, Extruder RPM 11%, |
| L01 non-woven material | Melt Pump RPM 18% |

Similar results (not shown) to those in Table 9 were obtained with polypropylene based on the guidance provided by the present specification for those of ordinary skill in the art.

### Example 7

### Antimicrobial Efficacy of Non-Woven Fabric

The standard method for analyzing the efficacy of microbial challenge testing for an antimicrobial article is to run the active samples side by side with an untreated control, in accordance to AATCC100.

From a stock plate prepared on organism specific media, inoculate a sufficient number of 10 ml tubes of pre-reduced growth media using an isolated colony, mix, and incubate at 35-37° C. for 24±2 hours. Following incubation, inoculate each of a minimum of 10 Agar plates with 100 μl of the broth culture. Spread the inoculum evenly with a sterile plate spreader (or equivalent). Invert plates and incubate for 7-10 days at 36±1° C. Anaerobic jars are recommended for use to prevent desiccation. Harvest growth from each plate by adding 5 ml of Phosphate Buffered Saline (PBS)+0.1% Tween 80 to each plate and gently scraping with a cell scraper or other appropriate device avoiding the collection of agar fragments where possible. Pool the suspension into sterile 50 ml conical tubes.

A suspension of each test organism was exposed to 1″×1″ Biovation provided product samples (test carrier) for the specified exposure time. A single (1″×1″) stainless steel control carrier was inoculated with the test organism and was exposed for 24 hours. After exposure, the test and control carriers were transferred to neutralizer and assayed for survivors. Appropriate culture purity, carrier sterility, Triton X-100 sterility, neutralizer sterility, stainless steel and neutralization confirmation controls were performed.

Inoculate each test and untreated carrier, at staggered intervals, with 0.01-0.03 ml (10-30 μL) of prepared organism suspension using a calibrated Pipettor or sterile 4 mm i. d. loop. Expose all test/untreated carriers (with exception to applicable T₀ carriers) at the desired exposure temperature for the duration of the exposure time(s).

Immediately after inoculation, transfer each T₀ (time zero) test/untreated carrier into 10-100 ml of neutralizer (representing a 10° dilution). Mix each carrier using an appropriate method (i.e. vortex mixing, sonication etc). Prepare serial dilutions of the neutralized solution and plate 1.0 ml aliquots of the 10° to 10 dilutions, in duplicate, using standard spread plate technique. If swarming is a concern, plate 1.0 ml of a 10° and 0.1 ml of 10° through 10⁻³ in duplicate.

At each Biovation specified exposure time (T₂₄ for the purpose of this test protocol), transfer each test/untreated carrier into 10-100 ml of neutralizer (representing a 10° dilution). Mix each carrier using an appropriate method (i.e. vortex mixing, sonication etc). Prepare serial dilutions of the neutralized solution and plate 1.0 ml aliquots of the 10° to 10⁻³ dilutions, in duplicate, using standard spread plate technique. If swarming is a concern, plate 1.0 ml of a 10° and 0.1 ml of 10° through 10⁻³ in duplicate, as above.

Incubate the test plates and control subcultures at 35-37° C. for 48±4 hours.

If necessary, subcultures may be stored for up to 3 days at 2-8° C. prior to examination. Following incubation (or incubation and storage), the plates and controls will be visually examined for growth and enumerated. Representative test subcultures showing growth may be subcultured, stained and/or biochemically assayed to confirm or rule out the presence of the test organism. If possible, subcultures containing 30-300 colonies will be used for calculations.

The data provided in Table 10 affords the antimicrobial efficacy of the blood pressure cuff non-woven at T₂₄ (time interval of 24 hours) against the following microorganisms: MRS. aureus, P. aeruginosa, K. pneumoniae, C. albicans, VRE, C. difficile and A. baumannii. It is customary to report efficacy as a log₁₀ reduction (% reduction can also be reported) of the microorganism by the active article at the time-frame of interest (T₂₄ in this screening scenario) by comparing the log₁₀ (untreated Control) CFU(s) at 24 hours to the log₁₀ (active sample) surviving CFU(s) at 24 hours for each challenging microorganism according to the following equation:

Average Log₁₀(CFU Untreated Control)@T₂₄−Average Log₁₀(CFU Active Sample)@T₂₄=Log₁₀ reduction@T₂₄ Equation1:

TABLE 10

| | Organism | Organism | | Percent |
|---|---|---|---|---|
| Test Article | Count (CFU/mL) - | Count (CFU/mL) - | | Reduction |
| Identification | Zero Time | 4 Hour | Log Reduction | (%) |
| TP02102013 - | 2.30 × 10⁵ | <1.00 × 10² | 5.48 | >99.999 |
| MRSA | | | | |
| Control -MRSA | 2.88 × 10⁵ | >3.00 × 10⁷ | | |
| TP02102013 - K. pneumoniae | 1.02 × 10⁵ | <1.00 × 10² | 5.48 | >99.999 |
| (Kp) | | | | |
| Control -Kp | 1.03 × 10⁵ | >3.00 × 10⁷ | | |
| TP02102013 - P. aeruginosa | 2.73 × 10⁵ | <1.00 × 10² | 5.48 | >99.999 |
| Control -Pa | 2.23 × 10⁵ | >3.00 × 10⁷ | | |
| TP02102013 - | 3.30 × 10⁵ | <1.00 × 10² | 5.48 | >99.999 |
| VRE | | | | |
| Control -VRE | 4.30 × 10⁵ | >3.00 × 10⁷ | | |
| TP02102013 - A. baumannii | 3.00 × 10⁵ | <1.00 × 10² | 5.48 | >99.999 |
| (Ab) | | | | |
| Control -Ab | 2.90 × 10⁵ | >3.00 × 10⁷ | | |
| TP02102013 - C. albicans | 2.53 × 10⁵ | 1.25 × 10² | 5.38 | >99.999 |
| (Ca) | | | | |
| Control -Ca | 3.56 × 10⁵ | >3.00 × 10⁷ | | |
| TP02102013 - C. difficile | 1.15 × 10⁷ | 1.69 × 10⁵ | 1.83 | 98.5 |
| (Cd) | | | | |
| S.S. Control - Cd | | 1.16 × 10⁷ | | |

### Example 8

### Measuring Silver and Copper Antimicrobial Content in PLA Non-Woven Material Layer

The analysis of solid samples for elements such as silver or copper has been much studied and each was found to have some liabilities or difficulties. Methods such as wavelength dispersive X-ray fluorescence spectroscopy (WD-XRFS), laser ablation inductively coupled plasma mass spectrometry (LA-ICPMS) as well as conventional acid digestion in a Kjeldahl flask in combination with dry ashing and microwave assisted digestion followed by atomic absorption spectrometry (AAS) are the “go to” analytical tools especially for biological and environmental samples. However, solid sample analysis affords some challenging issues for each of the aforementioned methods as described in F. Vanhaeke, et al, Spectrochimica Acta: Part B 62, (2007) pp 1185-1194. For example, this study showed LA-ICPMS has potential for the direct analysis of solid samples but for variations in ablation efficiency which affords calibration difficulties. Similar calibration issues arise with WD-XRFS, mainly due to differences in absorption efficiency of X-rays. These authors describe having obtained accurate results for Ag determination using conventional acid digestion in a Kjeldahl flask in combination with dry ashing and microwave assisted digestion followed by AAS. Occasionally however, they noted analyte losses and/or incomplete dissolution as the source(s) of discrepancy.

The reagents and materials for experimentation were as follows. As specified by good lab practice, only high purity reagents were employed in sample preparation. A Millipore (Billerica, Mass.) Milli-Q system was used to generate water of 18 MΩ purity. Concentrated nitric acid (HNO₃) and 30% hydrogen peroxide (H₂O₂) were obtained from Fisher Chemical (Houston, Tex.) and (1 mg/mL) Ag in HNO₃ was obtained from Acros Organics/Thermo Fisher Scientific (Geel, Belgium and Boston, Mass.) for sample digestion and calibration standard preparation, respectively. The non-woven material with silver antimicrobial was manufactured as exemplified above.

For the digestion of PLA non-woven samples, we used a HotBlock Pro Digestion System from Environmental Express (Charleston, S.C.). The 54-well HotBlock Pro for 50 mL samples has an external thermocouple and an external controller to monitor and record sample temperatures. The controller also allows you to program and implement the digestion method (see below). For analysis of samples by Atomic Absorption Spectrometry, an ICE 3000 Series Flame AA Spectrometer from Thermo Fisher Scientific (West Palm Beach, Fla.) was used. The silver (Ag) hollow cathode lamp was purchased separately from Thermo Fisher Scientific (West Palm Beach, Fla.)

For digestion, we employed an adaptation of EPA Method 3050B for use with the Environmental Express HotBlock Digestion System. The 0.5 g samples were each placed into a 50 mL borosilicate digestion vial to which 5 mL of a 1:1 mixture of concentrated HNO₃ and 18 MΩ water is post added. The digestion vials were placed into the HotBlock unit, affixed with reflux caps and heated at 95° C. for 15 min. Samples were allowed to cool and an additional 5 mL of concentrated HNO₃ was added and then heated @95° C. for 30 min. This step was repeated until no brown fumes were given off by the samples. The samples were then heated for an additional 1.5 hours after which they were removed from the HotBlock Pro and completely cooled. To each of these vials was added 2-5 mL of 18 MΩ water and 0.5 mL of 30% H2O2 slowly. An exothermic reaction was allowed to occur for approximately 5-10 minutes and the samples were placed back in the HotBlock with the ribbed watch glasses in place. Effervescence was controlled by lifting the samples out of the HotBlock while allowing the reaction to continue. Care was taken to ensure that the samples did not overflow the vials. H₂O₂ was continually added in 0.5 mL increments until the sample remained unchanged in color (no longer than 30 minutes). Then heating was continued for a total of 2 hours.

For the analysis of samples for Flame AA, 5 mL of concentrated hydrogen chloride (HCl) was added to each sample and covered with a ribbed watch glass and heated to reflux at 95° C. for 15 minutes. After cooling completely, the samples were diluted to 50 mL with 18 MΩ water. A calibration curve was constructed on the basis of absorbance obtained for aqueous standards containing 0.5 ppm, 10 ppm, and 50 ppm Ag in solution.

Two identical sets of samples were tested to account for repeatability; they are denoted as “A” and “B” in the testing protocol.

The sample weights and composition of materials is shown in Table 11 below. MB23 is a master-batch with of 20% silver Zeolite grade AC-10D from AgION with 80% PLA; whereas MB22 is a masterbatch with 20% silver glass grade WPA Ionpure® from Marubeni/Ishizuka with 80% PLA.

TABLE 11

| Sample # | Sample Information | Weight of A (g) |
|---|---|---|
| 1 | 16% MB21 & 16% | 0.051 |
| | MB23 | |
| 2 | 16% MB21 & 16% | 0.057 |
| | MB23 | |
| 3 | 16% MB21 & 16% | 0.082 |
| | MB23 | |
| 4 | 16% MB21 & 16% | 0.072 |
| | MB23 | |

The results obtained from the analysis of these samples run in triplicate are presented in Table 13. These results are expressed in ppm Ag. The expected Ag content, presented in Table 12, has been calculated based upon the type of silver (WPA Ionpure® or AgION® and the amount added during processing. We observed good agreement between the theoretical values and the analytical results with the exception of the copper concentrations which were slightly elevated from the maximum range limit.

Table 12 is shown below for theoretical Ag calculations. Because the silver zeolite (AgION) has a range of 2%-5% pure silver content and 4%-7% pure copper content, the theoretical calculations for Samples 1-4 are denoted as a range.

TABLE 12

| | Copper Concentration | Silver Concentration |
|---|---|---|
| Sample # | (ug/g) | (ug/g) |
| 1 | 1280-2240 | 1280-2240 |
| 2 | 1280-2240 | 1280-2240 |
| 3 | 1280-2240 | 1280-2240 |
| 4 | 1280-2240 | 1280-2240 |

Table 13 is shown below to demonstrate the Ag & Cu concentrations gathered by Flame AA.

TABLE 13

| Sample | Copper (ug/g) | Silver (ug/g) |
|---|---|---|
| 1 | 3204.24 | 2489.77 |
| 2 | 2411.70 | 2047.06 |
| 3 | 2308.12 | 1726.06 |
| 4 | 2974.97 | 2185.56 |

The above values for silver and copper concentration are reflective of the antimicrobial activity document in Table 10.

### Example 9

### Incorporation of Co-Polyester Barrier Film with the Non-Woven PLA

The rolled non-woven is then unwound, and passed through a series of lamination rolls. The non-woven is laminated to Bioflex 235-02, a flexible barrier film, composed of a thermoplastic co-polyester extruded film from Scapa (Windsor, Conn.), using a double coated acrylic pressure sensitive adhesive film, Dublfilm SP357E from Scapa (Windsor, Conn.), in order to impart strength and fluid impermeability of the shield. The Bioflex 235-02 co-polyester film, with a 2 mil thickness, has a tensile strength of 1600 lbs/in² and 500% elongation measured in accordance of ASTM D5035. The mechanical properties for the composite PLA, adhesive, and barrier film, is 15.32 lbs/in² with a 35.7% elongation, in accordance with ASTM D5035.

### Example 10

### Incorporation of Polyurethane Barrier Film with the Non-Woven PLA

Similar to the embodiment of Example 4, the rolled non-woven is laminated to Bioflex 130-02, a flexible barrier film, composed of medical grade thermoplastic polyurethane extruded film from Scapa (Windsor, Conn.), using a double coated acrylic pressure sensitive adhesive film, Dublfilm SP357E from Scapa (Windsor, Conn.), in order to impart strength and fluid impermeability of the shield. The Bioflex 130-02 polyurethane film, with a 2 mil thickness, has a tensile strength of 7000 lbs/in² and 500% elongation. The mechanical properties for the composite PLA, adhesive, and barrier film, is 15.50 lbs/in² with a 43.92% elongation, in accordance with ASTM D5035. The moisture vapor transmission rate of the composite (non-woven and barrier film) is 7.98 g/h*m². The determination of permeation is conducted according to ASTM E96/E96M-10, Water Vapor (moisture vapor) Transmission of Materials Test methodology using permeation cups by BYK-Gardner (Columbia, Md.) and weigh scale by Mettler Toledo (Columbus, Ohio).

### Example 11

### Adhesive Backing for Adhering the Shield to the Blood Pressure Cuff

Dublfilm SP357E double coated adhesive is applied to the barrier film of the composite (non-woven and barrier film), by way of an island placement module, in order to secure the shield to the blood pressure cuff. In the present embodiment, the composite created in Examples 3 and 4, is sheeted to the following dimension 6.5″ width and 17″ length. Two 1″×8″ strips of Dublfilm SP357E pressure sensitive adhesive film, sourced from Scapa (Windsor, Conn.), are applied length wise to the barrier film. The strips are positioned 0.5″ from the length edge and width edge, and 3.5″ gap remains between the inner edges of the adhesive strips.

### Example 12

### Adhesive Backing for Adhering the Shield to the Blood Pressure Cuff

Similar to Example 7, Dublfilm SP357E double coated adhesive is applied to the barrier film of the composite (non-woven and barrier film), by way of an island placement module, in order to secure the shield to the blood pressure cuff. In the present embodiment, the composite created in Examples 3 and 4, is sheeted to the following dimension 6.5″ width and 17″ length. One rectangular island adhesive block 5″×8″ of Dublfilm SP357E pressure sensitive adhesive film, sourced from Scapa (Windsor, Conn.), is applied length wise to the barrier film. The island adhesive rectangle is positioned 0.75″ from the length edge and 0.5″ width edge, so that the 8″ length of the block runs lengthwise with the shield.

### Example 13

### Ease of Removal with No Adhesive Residue

The blood pressure cuff shield of the current invention was tested for ease of removal as well as for the absence of adhesive residue on the cuff from which it was removed. A 180 degree peel test was designed for use with an EJA Thwing Albert tensile tester (West Berlin, N.J.) to test the force necessary to remove the shield from a reusable nylon blood pressure cuff shield. The values obtained from the Dublifilm SP357E adhesive strips were compared with adhesive on Scotch Tape and Duck Brand duct tape.

A composite similar to that of Examples 10 & 11 (PLA laminated to barrier film) was created, Dublifilm SP357E double sided adhesive was applied by hand to the barrier film side of the adhesive. The composite was cut into 1″×9″ strips. The comparative adhesive products were cut into 1″×9″ strips.

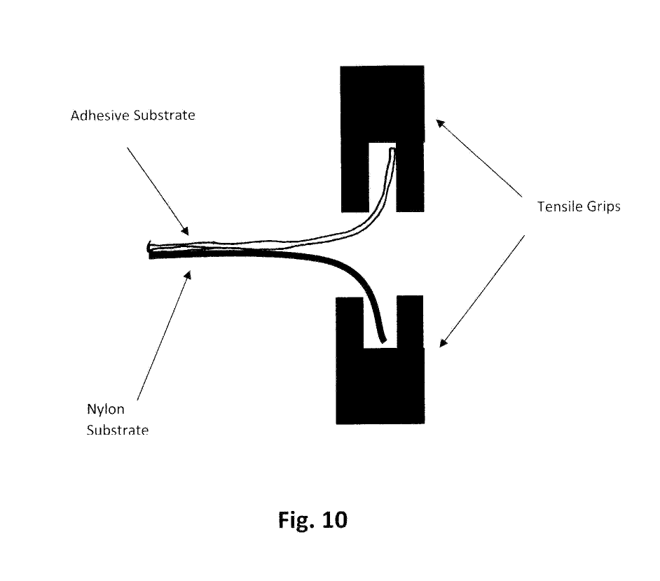

A 1″×9″ strip of nylon was cut from an ADC reusable blood pressure cuff. The adhesive sample was lined up to one short edge of the nylon while leaving 1 inch of the opposing nylon end un-adhered to the adhesive substrate in order to secure within the grips of the EJA Thwing Albert tensile tester, please refer to FIG. 10.

The EJA Thwing Albert tensile tester was calibrated according to ASTM D5035. The settings not described within the ASTM are as follows: gage length, 1 inch; test speed, 3.0 in/min. The MAP 3 software tabulated Breaking Force (lbs) for each adhesive substrate, presented in Table 14, below. Breaking force represents the maximum force applied to separate the adhesive substrate from the nylon sheet.

TABLE 14

| Adhesive Substrate | Breaking Force (lbs) |
|---|---|
| Scotch Tape | 0.050 |
| Composite w/ Dublifilm SP357E | 0.458 |
| Duck Brand Duct Tape | 0.710 |

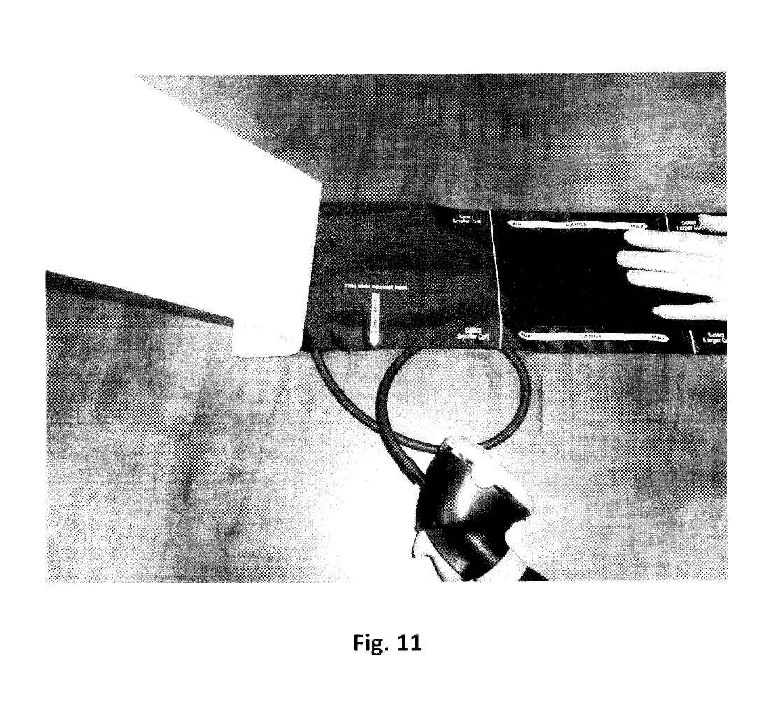

The above data, documented in Table 14, shows that the adhesive used to apply the blood pressure cuff shield to the blood pressure cuff is strong enough to secure the shield for a 24 hour period of use, but requires little effort to remove. As a comparison, two well-known adhesive substrates were tested alongside of the composite with Dublifilm SP357E. Table 14 demonstrates that the composite with Dublifilm SP357E is easier to remove than duct tape but has a stronger bond to the nylon substrate compared to scotch tape. FIG. 11 shows that no residue from the Dublifilm SP357E adhesive is left behind on the blood pressure cuff upon removal of the shield.

### Example 14

### Conformability of Blood Pressure Cuff Shield

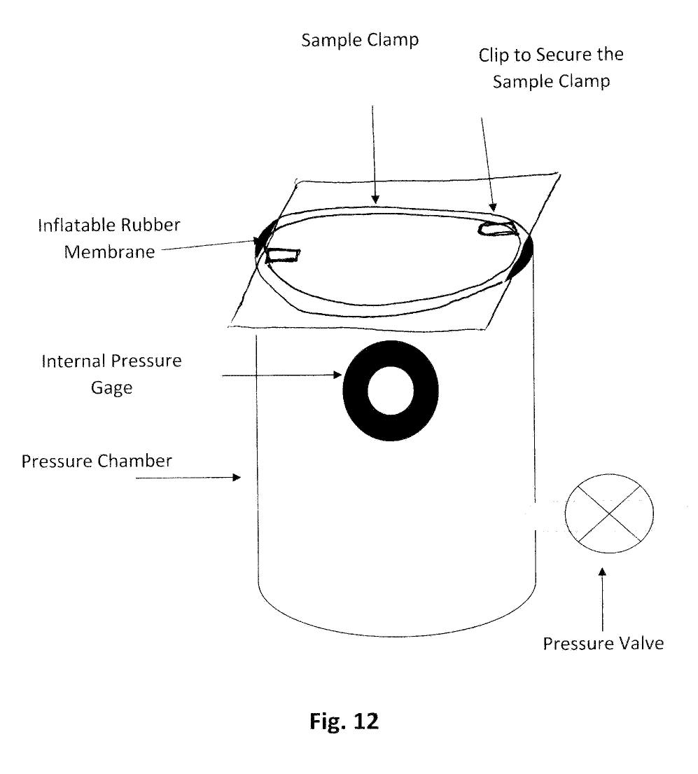

Conformability was analyzed by way of an amended version of the Queens Methodology, where a conformability apparatus used is to impart a set pressure on rubber membrane which transfers to the sample substrate, ballooning the substrate, from which the height of deformation is then measured. A conformability apparatus was crafted specifically to our needs at the Biovation facility, please refer to FIG. 12. The Samples were cut to size, 6″×6″, and secured on top of the conformability apparatus with the PLA non-woven side down simulating direction for contact with the skin. The pressure of the apparatus was adjusted to 206 mmHg. The height of deformation of the substrate was measure. The blood pressure cuff shield composite was compared to a 3.5 mil release liner (Scapa, Windsor, Conn.) and a KimWipe® (Kimberly Clark, Irving, Tex.).

The blood pressure cuff shield PLA composite conformed with the rounded form of the inflated conformability apparatus similarly to the KimWipe, where as the release liner began to ripple and buckle when the apparatus was inflated to 206 mmHg.

Table 15, below, shows the height of deformation measured for the blood pressure cuff shield composite, KimWipe®, and the release liner. The height of deformation shown in Table 15 proves that the blood pressure cuff shield contours to shapes easily, in comparison with a 3.5 mil release liner.

TABLE 15

| Substrate | Height of Deformation (cm) |
|---|---|
| KimWipe ® | 2.3 |
| Blood Pressure Cuff Shield Composite | 1.9 |
| Release Liner | 1.1 |

1. An antimicrobial, biodegradable blood pressure cuff shield, comprising:
one or more layer of non-woven fibers comprising one or more biodegradable thermoplastic polymers and one or more antimicrobial agent selected from the group consisting of silver-based, silver ion-based, copper-based, non-silver based, and non-copper based antimicrobial agents, including means for attaching the blood pressure cuff shield to a blood pressure cuff.

2. The antimicrobial, biodegradable blood pressure cuff shield according to claim 1, wherein the one or more layer of non-woven fibers is laminated to a non-porous barrier film, the non-porous barrier film including means for attaching the blood pressure cuff shield to a blood pressure cuff.

3. The antimicrobial, biodegradable blood pressure cuff shield according to claim 1, wherein the non-woven fibers are selected from the group consisting of PolyLactic Acid (PLA);
PolyCaproLactone (PCL) and PolyHydroxyAlkanoate (PHA), polypropylene, polyethylene, and blends, alloys and copolymers thereof.

4. The antimicrobial, biodegradable blood pressure cuff shield according to claim 2, wherein the non-porous barrier film comprises a material selected from the group consisting of a biaxially oriented polypropylene, compostable polystyrene, thermoplastic polyurethane, thermoplastic polyolephin blends, polyamides, PLA, and thermoplastic copolymers thereof.

5. The antimicrobial, biodegradable blood pressure cuff shield according to claim 4, wherein the thermoplastic copolymer is a thermoplastic co-polyester.

6. The antimicrobial, biodegradable blood pressure cuff shield according to claim 1, wherein the silver and silver ion-based agents are selected from the group consisting of silver halides, nitrates, nitrites, selenites, selenides, sulphites, sulphates, and sulphadiazines, silver polysaccharides, silver zirconium complexes, organic-silver complexes, including silver trapped in or by synthetic, natural or naturally-derived polymers, and inorganic or organic compound structures containing silver as part of the structure and mixtures thereof.

7. The antimicrobial, biodegradable blood pressure cuff shield according to claim 1, wherein the copper and copper ion-based agents are selected from the group consisting of copper halides, acetates, carbonate, nitrates, nitrites, selenites, selenides, sulphides, sulphates, and sulphadiazines, copper polysaccharides, copper zirconium complexes, and mixtures thereof.

8. The antimicrobial, biodegradable blood pressure cuff shield according to claim 1, wherein the non-silver and non-silver ion-based agents are selected from the group consisting of compounds containing zinc, copper, titanium, magnesium, quaternary ammonium, silane (alkyltrialkoxysilanes) quaternary ammonium cadmium, mercury, biguanides, amines, glucoprotamine, chitosan, trichlocarban, triclosan (diphenyl ether (bis-phenyl) derivative known as either 2,4,4′-trichloro-2′ hydroxy dipenyl ether or 5-chloro-2-(2,4-dichloro phenoxyl) phenol), aldehydes, halogens, isothiazones, peroxo compounds, n-halamines, cyclodextrins, nanoparticles of noble metals and metal oxides, chloroxynol, tributyltins, triphenyltins, fluconazole, nystatin, amphotericin B, chlorohexidine, alkylated polethylenimine, lactoferrin, tetracycline, gatifloxacin, sodium hypophosphite monohydrate, sodium hypochlorite, phenolic, glutaraldehyde, hypochlorite, ortho-phthalaldehyde, peracetic acid, chlorhexidine gluconate, hexachlorophene, alcohols, iodophores, acetic acid, citric acid, lactic acid, allyl isothiocyanate, alkylresorcinols, pyrimethanil, potassium sorbate, pectin, nisin, lauryl arginate, cumin oil, oregano oil, pimento oil, tartaric acid, thyme oil, garlic oil (composed of sulfur compounds such as allicin, diallyl disulfide and diallyl trisulfide), grapefruit seed extract, ascorbic acid, sorbic acid, calcium compounds, phytoalexins, methyl paraben, sodium benzoate, linalool, methyl chavicol, lysozyme, ethylenediamine tetracetic acid, pediocin, sodium lactate, phytic acid, benzoic anhydride, carvacrol, eugenol, geraniol, terpineol, thymol, imazalil, lauric acid, palmitoleic acid, phenolic compounds, propionic acid, sorbic acid anhydride, propyl paraben, sorbic acid harpin-protein, ipradion, 1-methylcyclopropene, polygalacturonase, benzoic acid, hexanal, 1-hexanol, 2-hexen-1-ol, 6-nonenal, 3-nonen-2-one, methyl salicylate, sodium bicarbonate and potassium dioxide.

9. The antimicrobial, biodegradable blood pressure cuff shield according to claim 1, wherein the one or more layer of non-woven fibers is calendared.

10. The antimicrobial, biodegradable blood pressure cuff shield according to claim 1, wherein the means for attaching the blood pressure cuff shield to a blood pressure cuff is a gentle release adhesive that adheres to the blood pressure cuff for up to 48 hours and upon removal does not leave any by-products or residue.

11. The antimicrobial, biodegradable blood pressure cuff shield according to claim 10, wherein the adhesive is selected from the group consisting of silicone, polyurethane, and pressure sensitive adhesives.

12. The antimicrobial, biodegradable blood pressure cuff shield according to claim 1, wherein the one or more layer of non-woven fibers is biobased.

13. The antimicrobial, biodegradable blood pressure cuff shield according to claim 1, wherein the one or more layer of non-woven fibers is hydrophobic or hydrophilic.
